(19) United States
(12) Patent Application Publication
(10) Pub. No.: US 2023/0148923 A1
(43) Pub. Date: May 18, 2023

(54) DEVELOPMENT AND IMPLEMENTATION OF PSYCHOLOGICAL STATE MODEL

(71) Applicant: Cerebral Therapeutics, Inc., Aurora, CO (US)

(72) Inventors: Daniel J. Abrams, Denver, CO (US); Aaron Reite, Aurora, CO (US); Martin Lawrence Reite, Greenwood Village, CO (US)

(21) Appl. No.: 17/974,042

(22) Filed: Oct. 26, 2022

Related U.S. Application Data
(60) Provisional application No. 63/280,367, filed on Nov. 17, 2021.

(51) Int. Cl. A61B 5/16; A61B 5/00; A61B 5/37; A61B 5/372; A61B 5/245

(52) U.S. Cl. CPC A61B 5/165; A61B 5/0006; A61B 5/37; A61B 5/372; A61B 5/7267; A61B 5/7282; A61B 5/245; A61B 5/4836; A61B 5/7275

(57) ABSTRACT

A method receives continuous EEG data for a long duration of time from at least one electrode intracranially implanted in a subject. The method determines a current or predicted brain state from the EEG data using an artificial intelligence (AI) model.

### RELATED APPLICATION

This application claims the benefit of U.S. Provisional Patent Application No. 63/280,367, filed on Nov. 17, 2021, which application is hereby incorporated by reference in its entirety.

### FIELD

The present technology is generally related to methods, devices, and systems to understand, monitor, or predict psychological brain states of a subject based on electroencephalogram (EEG) data and the development and implementation of artificial intelligence (AI) models of psychological brain states.

### INTRODUCTION

Brain activity is difficult to study because of the inherent complexity of the brain, which includes billions of neurons and trillions of synaptic connections that connect with each other in complex configurations a thousand times a second. Furthermore, access to the brain requires brain surgery and scalp access has profound data collection limitations.

Existing methods for studying brain activity, such as magnetic resonance imaging (MRI), functional MRI (fMRI), electroencephalogram (EEG), and magnetoencephalogram (MEG) recordings, as well as other imaging techniques, are unable to continuously measure high quality temporally relevant brain data over long periods of time or over many episodes of changing feelings and thoughts. Accordingly, such methods are not readily amenable to development of artificial intelligence (AI) models of complex brain states such as psychological brain states.

From the earliest days of EEG, signal to noise ratio has been an area of major concern. Brain electrical activity recorded from the scalp is of very low voltage, and brain generated voltages must traverse the brain, cerebrospinal fluid (CSF), meninges, the skull, and skin prior to reaching the recording site where they can be detected. In addition, numerous external and biological noise sources can produce significant artifacts that interfere with EEG recording and interpretation. Major external noise sources include power line interference, interference from nearby electrical sources, movements and disturbances in patient and recording environments, artifacts related to electrode skin interface issues, artifacts related to movements of electrode wires, and related issues. Biological noise sources include eye movements (usually high voltage, slow), tongue movements (frequency variable), muscle activity (fast activity), EKG activity (both sharp and slow activity).

Early on with EEG analog signals recorded by ink pens writing on moving paper, artifacts were observed visually by a technician running an EEG machine and observing the patient. Background noise of 50 Hz or 60 Hz from power lines was controlled by recording inside metal screen shielded rooms, and biological “noise” resulting from muscle activity (high frequency) and eye movements (low frequency) were dampened by high pass (limiting low frequency eye movement) and/or band pass filtering (limiting muscle artifact). Other extraneous environmental (e.g., movements, passing vehicles) and physiological (e.g., sweat artifact) noise could be limited in a similar fashion. Most artifacts of this nature had distinguishing characteristics that allowed them to be recognized by trained technicians.

EEG recordings had relatively limited usefulness other than identifying wake and sleep states, focal or generalized epileptiform activity, and generalized or focal slow activity that might characterize generalized (e.g., stroke, encephalopathy) or more focal brain disorders (e.g., brain tumors, localized trauma). With the advent of newer imaging strategies like MRI, fMRI, positron emission tomography (PET), and single-photon emission computerized tomography (SPECT), clinical use of EEG diminished except in the epilepsy, sleep and electroconvulsive therapy monitoring domains, where it remains central. The newer imaging strategies provide excellent spatial resolution, but by their nature are unable to incorporate high frequency electrical activity that may prove important to understanding critical aspects of brain information processing.

Electrodes associated with deep brain stimulation (DBS) leads have been used to record neuronal activity for use in treating a disease of a patient, such as epilepsy. Implanted DBS neurostimulation devices operatively coupled to the leads may use the recorded electrical signals to predict the onset or detect the occurrence of a seizure. The implanted DBS devices may then apply an electrical signal to brain tissue in which the electrodes are implanted to prevent or reduce the severity of a seizure.

The implanted DBS devices process data received by the electrodes. The implanted DBS devices may band pass filter shorter episodes of data received by the electrodes to remove data not relevant to detection or prediction of seizures. For example, very high frequency oscillations (VHFOs) in local field potentials could potentially be used by the DBS devices to define the origin of a seizure or predict if a seizure would happen. Accordingly, data not associated with VHFOs in local field potentials may be removed to facilitate processing of the data and prediction or detection of seizures for patients with epilepsy.

Electrodes of DBS leads capture high quality data having high signal to noise ratio. However, the implanted DBS devices employ the captured data in a limited manner. That is, the DBS devices monitor for presence of certain signals within a limited range (e.g., frequency and local), for a particular purpose (e.g., predict seizure), and within limited time windows. The implanted DBS devices for epilepsy are not configured to use multiple events, long term data baselines, and/or complex processed and derived data transformations which are integral to AI model derivation and utilization to improve seizure prediction; rather, they use strategies which focus on EEG morphology recognition in the periods immediately before a potential assumed seizure event to count seizures and in some cases to predict seizures. Further, DBS devices are not configured to detect patterns associated with other brain states or conditions, such as psychological brain states.

### SUMMARY

The present disclosure relates to, among other things, methods, devices, and systems that utilize AI models to identify, classify, and/or predict psychological brain states based on intracranial EEG (iEEG) data. The iEEG data may be continuous iEEG (ciEEG) data. The iEEG data may be captured for a long duration of time to capture multiple occurrences of a psychological brain state episode. iEEG may provide high quality data having a high signal to noise ratio. Labels may be associated with the iEEG data. The labels may be applied by, for example, a subject in which the iEEG electrode is implanted or a person or device monitoring the subject in which the iEEG electrode is implanted. The iEEG data, which may be labeled, may be used to train AI models, such as deep neural networks, to classify or predict psychological brain states from past or current iEEG data. Once an AI model has been trained to reach satisfactory baseline performance on iEEG for classifying or predicting psychological brain states (e.g., 50% or greater mean absolute forecasting error and/or accuracy) it may be deployed to aid in a variety of downstream automatic or human decision-making processes. Furthermore, these AI models derived from implanted long-term high-quality EEG may be applied and extended to data received by more noisy signals obtained from non-invasive sources, such as scalp-based EEG, MEG, or the like; and further fine-tuned for the non-invasive source domain.

Without intended to be bound by theory, the present inventors believe that psychological brain states are associated with signal patterns in the brain that are detectable in global electrical activity not within one or another specific functional area of the brain such as the hippocampus for memory or the occipital cortex for vision. That is, the signals may be captured by electrodes positioned in any intracranial location capable of detecting relevant global electrical EEG signals, and AI models may be developed to extract or identify the relevant patterns, provided that the signals used to develop the model are sufficiently high quality, high bandwidth, and are continuously obtained for a sufficiently long duration to capture multiple occurrences of the pattern associated with the psychological brain state. The iEEG signals may be unfiltered or may be collected with appropriate filtering and/or pre-processing. The iEEG data may be labeled corresponding to the occurrence of psychological brain states. An AI model may employ the labeled data to train or fine-tune the model by, for example, optimizing an objective function that penalizes incorrect label predictions on temporally associated iEEG data.

An AI model may be developed and trained from the data to extract, identify or predict psychological brain states. The AI model may comprise a deep neural network. The AI model may ingest raw or processed iEEG data, include multiple hidden layers, and may include an output layer that classifies or predicts a psychological brain state corresponding to the iEEG input.

Examples of psychological brain states that may be identified or predicted include, but are not limited to, general affect or mood, such as happy, sad, content, or the like; anxiety, post-traumatic stress, and related anxiety domain disorders; depression, bipolar disorder, mania, depression or mood spectrum disorders; addiction, obsession and ritualized obsessive and compulsive disorder or suicidal or obsessional thoughts; hallucinations; cognitive, memory and attentional processing disorders, and the like; or degrees thereof. The AI model may predict imminent changes in brain state from current data, such as, but not limited to, the prediction of onset of a traumatic memory recall, a manic episode, an obsessional thought, hallucination or suicidal thought, anxious feeling or the like in the future for a given subject or patient.

Preferably, the data for developing the AI model is obtained from intracranial electrodes that are implanted during a medically warranted procedure in which the subject's brain is accessed, such a brain surgery, ventriculostomy, implantation of a therapeutic cerebrospinal fluid (CSF) or vascular catheter or lead, craniotomy, or the like. The electrodes may be implanted during the medically warranted procedure so that the subject does not undergo multiple invasive brain procedures.

Once the AI model has been developed and trained using iEEG signals, the AI model may be deployed, applied, or further fine-tuned using high quality signals from within the brain or lower quality signals, such as signals obtained from scalp-based EEG or MEG outside of the brain.

### DEFINITIONS AND CONTEXT FOR DEFINED TERMS

All scientific and technical terms used herein have meanings commonly used in the art unless otherwise specified. The definitions provided herein are to facilitate understanding of certain terms used frequently herein and are not meant to limit the scope of the present disclosure.

As used herein, a “continuous” EEG data, such as continuous iEEG data is EEG data that is collected at a frequency of 0.5 Hz or greater. Preferably, continuous EEG data is collected at a frequency of 1 Hz or greater, such as 2 Hz or greater, 3 Hz or greater, 4 Hz or greater, 5 Hz or greater, or 10 Hz or greater. Lower frequency information may be derived from higher frequency data collection. Accordingly, higher frequency data collection may permit identification of high frequency patterns, as well as lower frequency patterns, and mixed frequency patterns. Preferably, continuous EEG data is collected over a long duration of time.

As used herein, a “long duration of time” of collection of EEG data is a duration that occurs over multiple occurrences of a psychological brain state episode. Preferably, a long duration of time of collection of EEG data is a duration that occurs over 5 or more occurrences of a particular psychological brain state episode. A long duration of time of collection of EEG data may be a duration that occurs over 10 or more occurrences, 20 or more occurrences, 30 or more occurrences, 40 or more occurrences, 50 or more occurrences, or 100 or more occurrences of a psychological brain state episode. The duration of time required to capture multiple occurrences of a psychological brain state episode will depend on the frequency of the occurrence of the episode. A long duration of time of collection of EEG data may a few days or more, a few weeks or more, or a few months or more. Preferably, a long duration of time of collection of EEG data for an implantable system to build an AI model is 24 hours or more, such as ten days or more, or one month or more. Once the AI model is built using either implanted or non-implanted EEG the duration may be, for example, two or more hours to a few days.

In embodiments, continuous iEEG data collected over a long duration of time is continuously collected. That is, the data is not repeatedly captured in smaller time periods, such as 5 minute periods, 10 minute periods, 15 minute periods, 30 minute periods, or 60 minute periods, but rather the data is capture continuously over the long duration of time.

iEEG may provide high quality data having a high signal to noise ratio. As used herein, “high signal to noise ratio” means a signal to noise ratio that is 10 times or more greater than a signal to noise ratio of scalp-based EEG (see, e.g., Ball et al, NeuroImage 46 (2009) 708-716). A high signal to noise ratio may have a signal to noise ratio that is 20 times or more greater than, 30 times or more greater than, 40 times or more greater than, 50 times or more greater than, 60 times or more greater than, 70 times or more greater than, 80 times or more greater than, 90 times or more greater than, or 100 times or more greater than a signal to noise ratio of scalp-based EEG.

As used herein, a “brain state” is a symptom or function of the brain that (i) involves multiple areas and neuronal networks of the brain and (ii) is reflected in brain activity. A brain state may be manifest in normal or abnormal brain activity. Motor function, speech function, visual function, somatosensory sensation function, smell function, and the like, in and of themselves, are not brain states. These functions, in and of themselves, impact smaller, more discrete, less distributed regions of the brain. For example, visual function involves the visual cortex, visual radiations, and optic tract. A brain state may, however, involve one or more of motor function, speech function, visual function, smell function, and the like. A brain state may involve activity in 2 or more Brodmann areas, 3 or more Brodmann areas, 4 or more Brodmann areas, 5 or more Brodmann areas, 6 or more Brodmann areas, 7 or more Brodmann areas, 8 or more Brodmann areas, 9 or more Brodmann areas, or 10 or more Brodmann areas.

As used herein, a “psychological brain state” is a brain state having a mental or emotional component. A subject is typically aware of their psychological brain state in their feelings and thoughts. Examples of psychological brain states include general affect or mood, anxiety, depression, addiction, obsession, suicidal thoughts, hallucinations, cognition, attention, post-traumatic stress, and the like, and degrees thereof. As an example, a traumatic memory may involve one area of the brain, but post-traumatic stress disorder (PTSD) is the impact of that memory which distracts attention, causes fear, and/or impacts cognitive function. Accordingly, PTSD involves or impacts multiple regions and neural networks of the brain.

Psychological brain states do not include seizure activity and/or motor activity alone. However, electrical brain activity associated with epileptic and/or motor activity may be relevant to a broader psychological brain state. In embodiments, an AI model that identifies or predicts a psychological brain state does not identify or predict epileptic activity and/or motor activity.

As used herein, a “psychological brain state episode” is a discrete occurrence of a psychological brain state or a discrete occurrence of a component of a psychological brain state. For example, an episode of PTSD may occur whenever a subject recalls a traumatic memory that distracts attention, causes fear, and/or impacts cognitive function. A psychological brain state episode may last seconds or longer, minutes or longer, hours or longer, days or longer, or weeks or longer. The duration of the psychological brain state episode may depend on the psychological brain state or the component thereof. Psychological brain states episodes of long duration may be parceled into components of shorter duration. For example, depression may last for multiple months to years. Depression has multiple and differing components that have differing time courses, such as inability to sleep for long periods, anhedonia, suicidal thoughts, and lack of interest, amongst others. Parceling and prioritizing these dimensional components individually and prioritizing study and identifying ones that recur and have shorter duration with a beginning and an end to isolate multiple episodes is one strategy for approaching a long duration brain state like depression, regardless of whether the depression is either bipolar or monopolar depression. A psychological brain state episode may be intentionally triggered by, for example, one or more of an auditory stimulus, a visual stimulus, a tactile stimulus, an olfactory stimulus, or the like. Another approach is in more of an experimental setting to trigger a feeling, for example anhedonia, by watching or listening to a musical or visual trigger and collecting enough of those episodes using ciEEG to allow the development and training an AI model.

As used herein, a “biomarker of a physiological brain state” is human understandable measure of a signal whose presence is indicative of a past, current, or future psychological brain state. The signal may be generated by an AI model. In some embodiments, human understandable brain activity data patterns corresponding to a current, past, or future brain state may be identified by the AI model with high sensitivity and specificity. In such cases, the brain activity data patterns are referred to as biomarkers of the psychological brain state. Existing AI visualization techniques from other domains, such as language processing or computer vision, may be employed to identify such brain activity patterns; e.g., class activation mapping, attention visualization, and similar techniques. Such biomarkers may be used to aid decision making by humans or machine algorithms, for example by physicians for diagnosis.

As used herein “brain network” is an anatomic term which means more than one brain area in more than on region or lobe of the brain that is connected anatomically and by electrochemical circuitry. A brain network may be responsible for a given thought, emotion, and/or behavior.

As used herein, an “AI model” is an Artificial Intelligence model: a mathematical algorithm implemented in a programming language that recognizes patterns from data and/or performs a task, either automatically or by learning from data in a supervised, un-supervised, semi-supervised, or self-supervised fashion. Examples of AI model tasks include categorizing data; predicting a corresponding real number, such as predicted time until the start of an event; and generating probable/possible output data of another type, such as the generation of a possible image being viewed by a subject given the temporally associated iEEG. An AI model is used herein synonymously with a machine learning or deep learning model and may comprise an artificial neural network.

As used herein, “deep learning” is a sub-field of machine learning that does not require expert feature engineering, but rather learns data features automatically from large quantities of data. Deep learning algorithms or models may comprise an artificial neural network having multiple hidden layers and many (e.g., thousands, millions, or billions) of learnable parameters. Example deep learning algorithms include convolutional neural networks, generative adversarial networks, recurrent neural networks, transformers, autoencoders, and deep reinforcement learning models.

As used herein, “labelling” is the act of associating data, or segments of data, with a category or real number. Labelling may be performed by a subject from which iEEG data is being collected. Labelling may be performed by a person observing the subject, the iEEG data, or a rendition of the iEEG data. Labelling may occur by automatic processing techniques including other AI algorithms or data collected from other devices. Where a brain state is associated with other physiological parameters, such as heart rate, pupil dilation, body temperature, sweating, and the like, labelling may occur through automatic detection of such other physiological parameters.

A label may be a time-stamped moment or interval corresponding to the occurrence, or future and/or past occurrence of a psychological brain state or set of brain states collected in data. For example, a label may denote that a particular psychological brain state started at time t and ended at time t+x, that one brain state will change to another in t seconds, or that a certain brain state last occurred t seconds ago. A label may be acquired through use of a human (e.g., a neurologist manually examining iEEG); or through automated means, such as using other time correlated devices such as an accelerometer, heart rate monitor or camera data (e.g., “smart” glasses or video camera observations), or the like.

As used herein, “supervised learning” is the training of an AI algorithm by using labeled data to classify or predict the correct outcomes accurately.

As used herein, “unsupervised learning” is the automatic clustering of associated data by an AI algorithm. The AI algorithm may not be trained, or it may be trained without the benefit of labels.

As used herein, “self-supervised learning” is the training of an AI model where the labels are derived from the qualities of the data itself, rather than from, for example, input from a human expert. For example, an AI model may be trained to generate the next 5 seconds of EEG data given the current 5 seconds of EEG data, or it may be trained to generate intentionally removed time segments collected from one or more electrodes.

As used herein, “semi-supervised learning” is the combination of unsupervised or self-supervised learning with supervised learning. Semi-supervised learning is typically used when few labeled data examples are available, in which case the model may be trained using unsupervised or self-supervised learning and subsequently fine-tuned using the available labeled data.

As used herein, “training” an AI model is the act of algorithmically feeding the model labeled or unlabeled data to improve the AI model. For example, training may improve the AI model at one or both of pattern recognition and its designated task. Training usually occurs by iteratively optimizing an objective function on the training data.

As used herein, “fine-tuning” an AI model refers to the process of starting with an AI model trained on given data for a given task and then subsequently re-training the AI model on differing data and/or for a different task.

As used herein, “transfer learning” is the act or implementation of fine-tuning.

As used herein, “objective function” is the mathematical implementation of a penalty for an incorrect prediction from an AI model. It is used synonymously with loss function. An objective function computes a real number from each prediction that corresponds to how close the prediction is to the corresponding datum's label. A value of close to or equal to 0 may be computed if the prediction matches the label perfectly, while values far from 0 may be computed if the AI model's prediction is incorrect.

As used herein, “intracranial” means within the skull of a subject. An intracranial electrode may be placed at any suitable location within the confines of a skull of a subject. For example, the intracranial electrode may be placed in the brain of the subject or on a surface of the brain of the subject. An intracranial electrode may be placed in brain parenchyma (“intraparenchymal”). An intracranial electrode may be placed within the cerebrum of a subject (“intracerebral”). An intracranial electrode may be placed within an intracranial blood vessel (“intravascular”). iEEG signals include signals obtained from electrodes positioned on the surface of the brain of a subject and within the brain of the subject, including electrodes placed intraparenchymally, intracerebrally, and intravascularly.

As used herein, an “external device” means a device that is external to a subject. That is, the device is not implanted in the subject.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The terms “coupled” or “connected” refer to elements being attached to each other either directly (in direct contact with each other) or indirectly (having one or more elements between and attaching the two elements). Either term may be replaced to “couplable” or “connectable” to describe that the elements are configured to be coupled or connected. In addition, either term may be modified by “operatively” and “operably,” which may be used interchangeably, to describe that the coupling or connection is configured to allow the components to interact to carry out functionality.

As used herein, the term “configured to” may be used interchangeably with the terms “adapted to” or “structured to” unless the content of this disclosure clearly dictates otherwise.

The term “or” is generally employed in its inclusive sense, for example, to mean “and/or” unless the context clearly dictates otherwise. The term “and/or” means one or all the listed elements or a combination of at least two of the listed elements. The use of and/or” in some locations of the present disclosure is not intended to mean that the use of “or” in other locations cannot be interpreted as “and/or.”

The phrases “at least one of” and “one or more of” followed by a list refers to any one of the items in the list and any combination of two or more items in the list.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

### BRIEF DESCRIPTION OF THE DRAWINGS

FIG. 1 is a flow diagram illustrating an embodiment of a method for capturing and transmitting iEEG data.

FIG. 2 is a schematic diagram illustrating an embodiment of a system for collecting iEEG data, which may be used in establishing or refining an AI model.

FIG. 3 is a flow diagram illustrating an embodiment of a method for developing an AI model.

FIG. 4 is a graphical representation of EEG data over time and clusters of similar data provided by a self-supervised deep neural network trained on raw, unlabeled EEG data.

FIG. 5 is a graphical representation of a 2-D visualization of a self-supervised representation space, colored by sleep stages. Specifically, it is the output of a t-distributed stochastic neighbor embedding (tSNE) algorithm run on an embedding of EEG data taken from a latent space in a deep neural network.

FIG. 6 is a flow diagram illustrating an embodiment of a method for deploying an AI model.

FIG. 7 is a schematic block diagram illustrating an embodiment of a system that may collect data that may be used by an AI model to adjust therapy delivery.

FIG. 8 is plot illustrating relationships between electrical brain activity, brain states, and different types of treatment.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same as or similar to other numbered components

### DETAILED DESCRIPTION

The present disclosure relates to, among other things, methods, devices, and systems to understand, monitor, and predict psychological brain states of a subject based on electroencephalogram (EEG) data and the development and implementation of artificial intelligence (AI) models of psychological brain states. Developed AI models may identify, classify, and/or predict psychological brain states based on brain electrical activity. Identifying, classifying, and predicting psychological brain states includes identifying or predicting the frequency of occurrence of the brain state.

Initially, the AI model may be developed or trained using iEEG data. The AI model may be trained using iEEG data collected from an electrode intracranially implanted during a medically warranted procedure in which the subject's brain is accessed. The subject's brain may be accessed for any suitable purpose including a ventriculostomy, implantation of a vascular or CSF therapeutic catheter or lead, a craniotomy, or the like. The electrode may be implanted on the surface of the brain or dura, intraparenchymally, intravascularly (including within the arterial or venous side), or intracerebrally. Preferably, the electrode is placed in a minimally invasive manner relative to the medically warranted procedure.

The electrode may be an electrode associated with a device implanted in the brain of the patient during the medically warranted procedure, may be an electrode coupled to a device implanted in the brain such that implantation of the device results in implantation of the electrode, or may be an electrode that is separately implanted during the procedure. Examples of therapeutic devices that may be implanted in a brain of a subject include catheters, stents, and the like. The catheters may be used to deliver therapeutic agent to the brain, such as the brain parenchyma or to a cerebrospinal fluid (CSF)-containing space of the brain, to drain CSF from the brain, or the like. Examples of diseases that may be treated with a CSF drainage catheter include subarachnoid hemorrhage, intracerebral hemorrhage, subdural hemorrhage, extradural hemorrhage, obstructive hydrocephalus, nervous system cancer (such as secondary malignant neoplasm of the brain, spinal cord, or other parts of the nervous system; malignant neoplasm of the cerebellum nos; malignant lymphoma of an unspecified site, extranodal site, or solid organ site; and the like), cerebral artery occlusion, unspecified cerebral infarction, closed fracture of the base of the skull (which may be associated with one or more of subarachnoid hemorrhage, subdural hemorrhage, extradural hemorrhage, and loss of consciousness), infection and inflammatory reaction (such as due to nervous system device, implant or graft), mechanical complication of a nervous system device, implant or graft), traumatic brain injury, congenital hydrocephalus, aneurysms, and the like. The CSF drainage catheter may be an external ventriculostomy drainage catheter, a ventriculoperitoneal shunt, or the like. Examples of diseases for which an intravascular device may be implanted include placement of a stent in a blood vessel for stroke treatment or prevention or to incidentally treat motor diseases with brain stimulation. Diseases that may be treated by delivery of therapeutic agents to the brain via a catheter include epilepsy, bipolar disorder, depressive disorder spectrum, anxiety disorder spectrum including post-traumatic stress disorder (PTSD), cognitive disorder spectrum, memory disorder spectrum, processing speed disorder spectrum which may be associated with Parkinson's disease, Alzheimer's disease, dementia, Amyotrophic Lateral Sclerosis, Huntington's disease, lysosomal storage diseases, anxiety, depression, brain tumors, autism, autism spectrum disorder, stroke, schizophrenia, brain infection, and the like.

Leads having electrodes may be implanted alongside of catheters that may be inserted into a brain, such as a therapeutic agent delivery catheter or a CSF drainage catheter. In addition or alternatively, a catheter may be modified to include one or more recording electrodes. Examples of catheters modified to include recording electrodes or coupled to leads having recording electrodes are disclosed in U.S. patent application Ser. No. 17/380,415, filed on Jul. 20, 2021, which patent application is hereby incorporated herein by reference in its respective entireties to the extent that they do not conflict with the disclosure presented herein.

In embodiments, one or more electrodes are implanted on the surface of the brain or the dura or penetrate a small distance, such as 1 centimeter or less, into brain parenchyma. Such electrodes may be readily placed when the brain is accessed for another medical procedure. A lead may comprise the one or more electrodes. The lead or signal apparatus associated with the lead may, in some embodiments, be configured to record EEG signals without the ability to apply stimulation pulses to the brain tissue.

Regardless of the source, the electrodes are preferably placed and, along with any signal processing apparatus, are configured to continuously record and process electrical activity associated with global electrical brain activity for a long duration of time, rather than merely recording and processing local field potentials or electrical activity associated with a local region of the brain without the ability to determine more global brain activity for short periods of time. In addition or alternatively, multiple electrodes may be placed in different intracranial locations to collectively capture global electrical brain signals. One or more intracranial electrodes should be able to capture electrical activity data that is indicative of global brain activity or that may be processed to determine global brain activity.

An intracranial electrode may be placed in white matter of the brain. White matter is composed mainly of long-range myelinated axons and serves as a preferred target for monitoring global electrical activity of the brain. An electrode in the white matter may capture electrical activity associated with a psychological brain state rather than merely capturing signals localized to small regions of the brain. The high-quality brain activity signals recorded by electrodes in white matter may facilitate processing (e.g., interpretation and analysis) of the iEEG data.

An intracranial electrode may be placed in grey matter of the brain. Grey matter is composed mainly of neuronal cell bodies. An electrode in grey matter may primarily capture local field potentials, or electrical activity associated with local regions of brain. Recording activity within confined brain regions, or interactions between two or more grey matter regions may facilitate processing of the iEEG data.

Regardless of whether placed in white matter, grey matter, on the surface of the brain, in a blood vessel in the brain, or the like, electrodes may be placed in more than one location to better ensure that global electrical activity of the brain may be captured. Data captured between electrodes in different locations may be collated to facilitate processing of the iEEG data and development and training of the AI model.

The electrodes may be placed in any suitable location relative to the brain. Electrodes placed in any intracranial location should be able to be used to collect global brain activity data. Accordingly, location of implant of electrodes for use in developing AI models for psychological brain states may be much less relevant than for models for detecting or predicting seizures where detection of local or focal brain activity may be most relevant. Another example of an AI model that may require recording from a defined location in the brain, such as the amygdala or hippocampus, are models that may be developed using evoked potentials following deep brain stimulation (DBS). Unlike such models, the psychological brain state models described herein may be initially developed using iEEG data from an electrode positioned in any intracranial location.

In embodiments, recording electrodes for capturing continuous long duration iEEG signals as described herein are not electrodes of a lead used for DBS.

As indicated above, the electrodes are preferably implanted in a relatively non-invasive manner as the subject is undergoing a procedure for which access to the brain is required. In some procedures, a burr hole may be created in the subject's skull during the procedure. In some embodiments, electrodes are inserted through the burr hole to be intracranially implanted. The electrodes may be placed on the surface of the brain or on the surface of the dura.

In some embodiments, one or more electrodes are coupled to, or are a part of, a catheter device that extends into the subject's cerebrospinal fluid (CSF). Examples of such devices include CSF therapeutic agent delivery catheters, CSF drainage (ventriculostomy) catheters, CSF-peritoneal shunts, and the like. The catheter, or a distal portion thereof, may be positioned in a cerebral ventricle, such as lateral ventricle, the cisterna magna, or any other suitable CSF-containing space. As the CSF catheter device is implanted, the electrodes may be implanted. The surgical path and trajectory of implantation of the catheter may determine the location in of the brain in which the electrodes may be implanted. In some embodiments, such as with a ventriculostomy drainage catheter or a ventriculoperitoneal (VP) shunt that may need to be replaced, the CSF catheter may be withdrawn from the patient without removing the electrodes. If the catheter is removed while leaving the intracranial electrodes in place, the subject is considered a subject who has “had” a catheter implanted, e.g., for a medically warranted procedure.

For example, if the distal portion of the catheter is implanted in a lateral ventricle, the electrodes may be positioned in the white matter between the frontal access of kochers point and the entry of the catheter into the frontal horn of the lateral ventricle. As another example, if the distal portion of the catheter is implanted in the lateral ventricle, the electrodes may be positioned in the white matter between the occipital parietal access at Keens point and the trigone of the lateral ventricle. As another example, if the distal portion of the catheter is implanted in the lateral ventricle, the electrodes may be positioned in the white matter between the occipital parietal access at Keens point and the trigone of the lateral ventricle.

In some embodiments, one or more electrodes may be coupled to, or may be a part of, an intracranial intravascular stent. Accordingly, the electrode may be implanted as the stent is implanted. Examples of brain locations in which an intracranial intravascular stent may be placed include the distributions of the middle cerebral artery, anterior cerebral artery or posterior cerebral artery.

In some embodiments, when the psychological brain state AI model is used to identify, classify, and/or to predict depression, the one or more electrode is positioned in an intracranial location other than the amygdala or the hippocampus.

The electrodes may remain intracranially within the subject for long durations of time, perhaps even permanently. Provided that suitable signal apparatus is operatively coupled to the electrodes to transmit signals derived from the electrodes, high quality continuous iEEG data may captured and transmitted for long durations of time. The electrodes may be implanted and high quality continuous iEEG signals captured for any suitable duration of time. For example, the high quality continuous iEEG signals may be captured for one day or more, 1 week or more, 1 month or more, or 1 year or more. Preferably, the high quality continuous iEEG signals are captured and transmitted as long as the electrodes and signal apparatus are implanted in the subject and not less than six weeks in most examples.

Signal apparatus may process, transmit, or process and transmit data regarding the signals recorded by the electrodes. The signal apparatus may comprise any suitable components, such as components configured to one or more of: amplify, digitize, filter, and transmit data regarding the EEG signals recorded by the electrodes. For example, the signal apparatus may comprise one or more of: an amplifier, an analog to digital converter, a band pass filter, an antenna, a processor, and a transmission coil. The signal apparatus may transmit raw or processed iEEG data derived from the electrodes. The raw or processed iEEG data may be indicative of global brain electrical activity. The raw or processed iEEG data may be filtered or unfiltered data. Preferably, the iEEG data is unfiltered for initial training and development of the initial AI model.

The signal apparatus may be configured to sample the signals from the electrodes at any suitable frequency. For example, the signal apparatus may be configured to sample the signals at a frequency of about 1 Hertz or greater, 10 Hertz or greater, 100 Hertz or greater, or 1,000 Hertz or greater. Preferably, the signal apparatus is configured to sample the signals at a frequency of about 1,000 hertz or greater. The signal apparatus may process the signal at any suitable bit depth, such as 4 bits, 8 bits, 16 bits or greater.

The electrodes and signal apparatus may be configured in any suitable manner. For example, the electrodes and signal apparatus may be configured in a differential or referential mode. In differential mode, the system comprises an active electrode, a reference electrode, and a ground. The signal difference between an active electrode and a reference electrode may be amplified. The reference electrode may be a common reference for more than one active electrode. The reference electrode is preferably positioned a substantial distance from an active electrode and from the ground. A differential mode recording may be achieved, for example, by using four electrode (two electrodes per amplifier) and an additional ground electrode. In differential mode, the system may be configured to detect small differences between electrode pairs and may be less likely to be affected by large artifacts originating near the ground electrode. However, the system may not be particularly effective at detecting larger common signals.

To detect larger common signals, the system may be configured in referential mode, which may also be referred to as single-ended mode. Referential mode may use a single active electrode per amplifier. In referential mode, the output of the active electrode is amplified relative to the ground electrode, as opposed to the reference electrode in differential mode. The ground is preferably placed a substantial distance from the active electrode, which may result in amplification of signals that affect larger parts of the brain. Referential mode may be sensitive to artifacts. Proper placement of the ground electrode may mitigate some issues associated with artifacts. To detect larger scale common signals, which may be more indicative of a psychological brain state, referential mode may be used while mitigating the effects of ground signal noise by careful consideration of electrode placement.

In some embodiments, some electrodes and associated signal apparatus are configured in referential mode and some are configured in differential mode.

The signal apparatus may be implanted in the subject at any suitable location. The signal apparatus may comprise a power source, such as a battery, which may be rechargeable, or may be wirelessly powered. If the signal apparatus is wirelessly powered, the signal apparatus preferably includes an inductive coil, solenoid, or other suitable components to be wirelessly powered by an external apparatus and to transmit data regarding the signals recorded by the electrodes to the external apparatus. The signal apparatus is preferably implanted at a location where it may inductively couple with a device external to the subject. For example, the signal apparatus may be positioned under the scalp of the subject near an ear of the subject. Such positioning may allow the external device to be comfortably worn on or around the ear of the subject to provide suitable inductive coupling to power the signal apparatus and to wirelessly transmit data regarding the signals recorded by the electrodes from the signal apparatus to the external device. The external device may then transfer the data directly to the cloud or via another device, such as a smart phone, a personal computer, or the like, which may then transfer the data to a server in the cloud, or the like.

The signal apparatus may be configured to continuously transmit iEEG data derived from an intracranial electrode. The signal apparatus may be configured to continuously transmit the iEEG data for a long duration of time. The signal apparatus may be configured to transmit relatively unfiltered data containing a broad amount of relevant brain signal. That is, the signal apparatus may transmit data regarding a majority of the captured iEEG data. For example, the iEEG data corresponding to the transmitted data may not have been bandpass filtered. As another example, the subsets of the iEEG data are not extracted for transmission. Rather, the majority of the iEEG data is transmitted by the signal apparatus. The signal apparatus may be configured to continuously transmit data for 1 day or more, 1 week or more, 1 month or more, or 1 year or more.

The external device may be configured to continuously receive iEEG data derived from an intracranial electrode. The external device may be configured to continuously receive the iEEG data for a long duration of time. The external device may be configured to continuously receive data for 1 day or more, 1 week or more, 1 month or more, or 1 year or more.

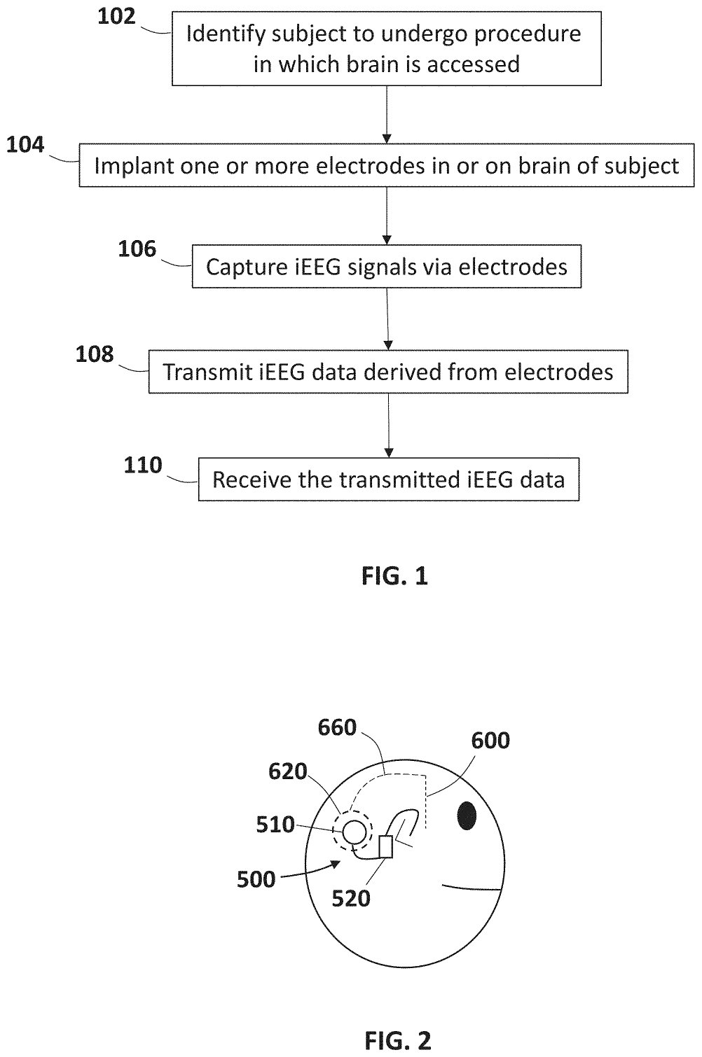

Referring now to FIG. 1, a method according to the present disclosure is illustrated. The method includes identifying a subject to undergo a procedure in which the subject's brain will be accessed (102). Such procedures include a ventriculostomy, implantation of a therapeutic catheter or lead, placement of an intracranial vascular stent, a craniotomy, and the like. The method also includes implanting one or more electrodes in or on the brain of the subject during the procedure in which the subject's brain is accessed (104). The electrodes may be implanted in a subject that is not undergoing a procedure in which the subject's brain is accessed. However, the subject would need to undergo a separate invasive procedure to intracranially implant the electrodes. The electrodes may capture iEEG signals from the subject's brain (106). The iEEG signals may be indicative of global brain electrical activity. The method includes transmitting iEEG data derived from the electrodes (108). The iEEG data may be raw or processed data. The iEEG data may be continuously transmitted for a long duration of time. The method may further include receiving the transmitted iEEG data (110). The iEEG data may be continuously received for a long duration of time.

Referring now to FIG. 2, an implantable system configured to record and transmit iEEG signals from the brain of a subject, as well as an external device 500 configured to receive the signals transmitted from the implanted system is shown. Implanted components are shown in dashed lines. The implanted system comprises a device 600, such as a catheter or lead comprising one or more electrodes (not shown), implanted in a subject's brain such that electrodes are implanted in the subject's brain. The electrodes may be implanted on a surface of the brain (not shown). The device 600 may include, or may be coupled to, a lead 660 that carries signals from the electrodes to signal apparatus 620. Alternatively, the signal apparatus may be in proximity to the electrodes (not shown). For example, a device may comprise both the signal apparatus and one or more electrodes.

The signal apparatus 620 may transmit data regarding the electrical signals to the external device 500. The external device 500 may comprise an inductive coupling component 510 that may be positioned over the signal apparatus 620 and comprises a processing component 520 operably coupled to the inductive coupling component 510. The processing component 520 may include, among other things, a rechargeable battery and a processor. The external device 500 may transmit data received from signal apparatus 620, or a processed version thereof, to suitable secondary device. The secondary device or external apparatus 500 may transmit data to the internet, where the data may be stored or retrieved by other computing devices as appropriate. In some embodiments (not shown), the signal apparatus may transmit data directly to the secondary device. Such other computing devices may establish or refine the AI model based on the iEEG data. In addition or alternatively, one or more of the signal apparatus 620, the external device 500, and the secondary device may, at least in part, establish or refine the AI model based on the iEEG data.

The use of intracranial electrodes and associated signal apparatus to transmit iEEG data derived from the electrodes allows for processing of large amounts of continuous, high-quality iEEG data over long durations of time to develop AI models that may identify, classify, and/or predict a psychological brain state.

Attempts to develop AI models of psychological brain states using EEG activity have largely been unsuccessful despite their success in epilepsy and sleep where scalp-based EEG recordings may be employed. However, above the scalp-based EEG recordings likely have too low of a signal to noise ratio to permit initial development of an AI model of a psychological brain state with a reasonable number of patients. As described herein, various AI techniques may be paired with a vast amount of high-quality iEEG data to train or develop an AI model to identify, classify, and/or predict a psychological brain state.

The AI models described herein may employ any suitable AI technique. For example, one or more of supervised learning, unsupervised learning, self-supervised learning, semi-supervised learning, and transfer learning techniques may be employed to train the model or to fine-tune the model. Such techniques may be particularly effective when large amounts of continuous iEEG data are available. The AI model may be established on or fine-tuned by data obtained from the brain of a single subject or a population of subjects.

The AI model may comprise a deep neural network (DNN); however, any suitable model architecture may be employed. A DNN is a machine learning algorithm which leverages a composition of many linear and nonlinear functions to map input data into a new desired output domain. The parameters of these functions are not directly designed by humans, but instead learned by optimizing a suitable objective function iteratively using vast quantities of labeled or unlabeled data. This allows the continual learning and improvement of a DNNs or other suitable AI model architecture performance through the collection of more high-quality data. The DNN or other suitable AI model architecture may be retrained at regular intervals, such as daily, weekly, monthly, etc. Furthermore, the specific DNN network architecture or other suitable AI model architecture may be upgraded or replaced to incorporate new future advancements in the field of artificial intelligence if new/future architectures are found to offer superior performance. DNNs have found widespread success across numerous domains that often match or surpass human performance on specific tasks.

Initially, machine learning strategies may be developed for iEEG data analysis associated with classifications for a psychological brain state including multi-layer perceptrons, convolutional neural networks, recurrent neural networks, generative adversarial networks, autoencoders, deep reinforcement learning, and/or transformers and the like. The initial strategies may utilize one or more of supervised learning, unsupervised learning, semi-supervised learning, and transfer learning approaches. The DNN or AI model architecture may include any technique from modern deep learning techniques as well as classical machine learning methods such as linear regression, logistic regression, support vector machines, nearest neighbors, decision trees, principal component analysis, naive Bayes classifier, k-means clustering; and bagging, boosting, and ensemble methods.

After an initial AI model has been sufficiently trained from long term high-quality iEEG data, the AI model (e.g., the learned parameters and algorithms) may be transferred and more meaningfully applied to lower quality external shorter-term (e.g., scalp) EEG recordings.

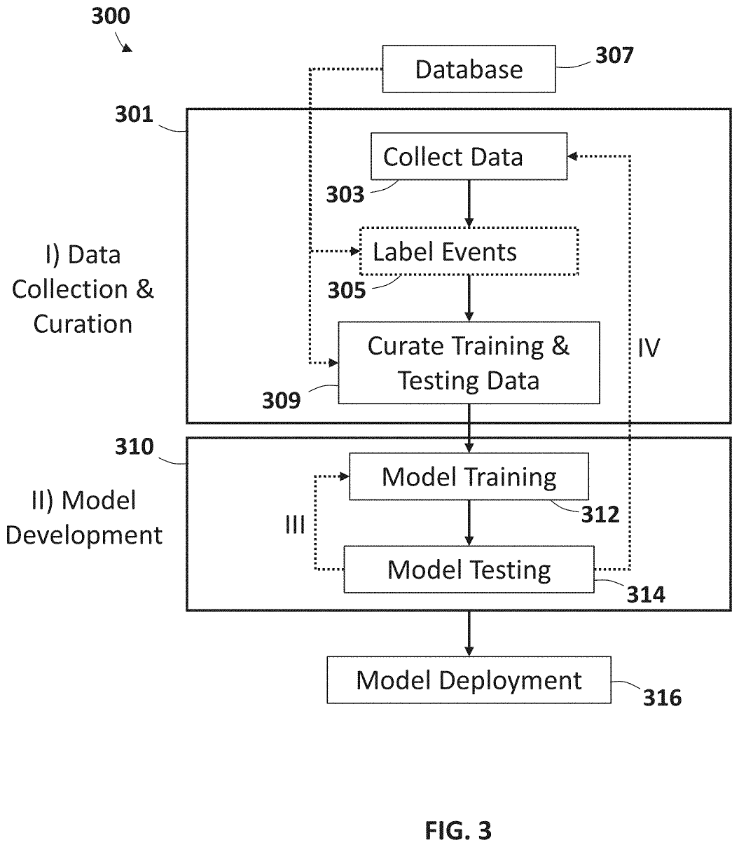

Referring now to FIG. 3, an example of a method 300 for developing an AI model is shown. To develop and AI model, data may be first developed and curated (I), as shown in block 301, which may involve collecting long duration continuous iEEG data in a suitable subject or groups of subjects, as generally depicted in block 303. The data may optionally be labeled as illustrated in block 305. Labeled data is needed for supervised learning but is not necessary for unsupervised or self-supervised learning. Optionally, data may be derived or supplemented from a database of collected data from different subjects and/or sensors, as generally illustrated in block 307. Once a sufficient quantity of data has been collected and labeled (as necessary), data may be curated and split into disjoint Training and Testing datasets as generally illustrated in block 309. Optionally, k-fold cross validation may be used so that all the data is available for training, yet testing is still performed on data that was not included in training.

Once data has been collected & curated (I) as illustrated in block 301, model development (II), as illustrated in block 310, may commence. Model development may involve selecting the model architecture, choosing an objective function and training hyperparameters, and optimizing the objective function iteratively over the training dataset, as generally illustrated in block 312. One or more of [un, self, semi, or fully]-supervised techniques may be employed. The model may then be tested against the test dataset as illustrated in block 314, and then deployed as illustrated in block 316. Optionally, e.g., if desired performance is not obtained, the model may be retrained with different choices of architecture, hyperparameters, and/or objective function (III), and/or additional data may be collected and curated prior to retraining (IV) to enhance performance.

Model development (II) as shown in block 310 may be performed by using transfer learning, in which the model in block 312 is initially pre-trained on other data and/or for other tasks. In transfer learning, the data may be EEG, MEG, or another suitable modality, and the model may be pre-trained on iEEG or another suitable modality.

Once the model is sufficiently developed, the model may be deployed as illustrated in block 316.

The method illustrated in FIG. 3 is one example of a method for developing an AI model for identifying, classifying, and/or predicting a psychological brain state. Any suitable method may be used to establish such an AI model. In general, such methods may include collecting data indicative of global brain activity, curating the data, and developing the AI model based on the collected and curated data. The data may be collected via any suitable device or system.

In some embodiments, a sensing device or system comprises a single recording electrode. In some embodiments, a sensing device or system includes multiple recording electrodes. By looking at impact on multiple recording electrodes in the brain at the same time, rather than a single electrode, data may be provided for relevant non-local electrical activity. That is, data more indicative of global brain activity may be collected. Data collected from multiple electrodes may represent a broader impact on the brain than data collected from a single electrode in a single location. Use of multiple electrodes may enable the AI model to evaluate information having higher degrees of freedom relative to use of a single electrode. For example, differences in EEG data obtained at different electrodes may be a contributing factor that allows an AI model to identify, classify, and/or predict a psychological brain state.

Regardless of the number of recording electrodes, the sensing device or system may collect, record, transmit, and/or receive data regarding brain activity.

Labeled data may be used as training data for certain AI models. Labeling may be performed by a physiological sensor, the subject from which iEEG data is generated, a person observing the subject or the subject's data such as a physician, or the like. Examples of physiological sensors that may be used to label data include heart rate monitors, sensors comprising cameras to determine extent of pupil dilation, body temperature sensors, skin conductance sensors, accelerometers to detect activity or motion, and the like. In one example, collected data may include continuous iEEG data of the subject's brain over time and labeled data may include the collected data and tagged (labeled) data indicative of a psychological brain state, such as when experiencing a recalled post traumatic memory or when experiencing mania, marked (labeled) at a time stamp or during a period of time relative to the iEEG data. The collected data may be labeled by marking. In some embodiments, a subject, physician, or the like may manually mark a psychological brain state observed, such as a state of elation or distress, for example, by pushing a button of a device in communication with a system in which the iEEG data is stored, collected, or processed. In some embodiments, sensor data may be used to mark the collected data.

Labeling can be done manually, by wearables, or other sensing devices. Examples of labeling input devices and sensing device that may be employed include, but are not limited to, smart phones, smart watches, visual/smart glasses, heart rate monitor, skin conductance monitor, respiration, temperature, or actimetry or accelerometer mobility and position monitor (e.g., configured to determine whether a subject falls, gets up from bed, goes for a walk, etc.). The AI model may use these additional data sources in several ways, including, but not limited to, using the additional data as auxiliary input or as training data in a pretext task; for example, it may be trained to predict the state of the additional data from concurrent iEEG. Non-limiting examples of labels may be for activities including arising from bed, going to the bathroom, starting a meal, or meeting a certain person or for emotions such as happy, elated, sad, or depressed. The labeling may be performed in real time, near real time, or retrospectively.

In some embodiments, labeling may be performed by time stamping. A subject may actuate a device to record the time whenever the subject has a feeling or thought or inclination for a behavior, such as a post-traumatic stress disorder (PTSD)-related memory recall or anxiety associated with that memory, bipolar-related starting to feel manic, or suicide-related feelings of suicide thoughts or obsessions starting. As the collected iEEG is also time stamped, the segment of iEEG concurrent with the time stamped label may be extracted.

Labelling may be done automatically and/or manually. Labeling may be turned into a rule or a series of rules that act as a filter for the collected data. In one embodiment, labelling may be performed for PTSD when a memory is triggered. For example, a memory may be triggered for a war veteran subject by seeing restaurant decorations including scenes of the country where the subject served (e.g., visual stimuli) or having similar smells from the same country (e.g., olfactory stimuli). A video-recording headwear (e.g., recording glasses) may be used to record those visual or olfactory stimuli to provide marking and labeling in a manual or automated manner. A video recording of the person may occur, and the person may change their activity because of distress thoughts. This event may be labeled to provide a time stamp for use in recognizing when an underlying event may have occurred and then that portion of the data may be analyzed as though an event was marked by the patient. When the visual stimulus is detected, the beginning and ending time may be used to label the onset or end or period of a psychological brain state without solely or initially relying on EEG data, such as the continuous iEEG data. This labeling of the data may add data points and data precision. Such a labeling technique could be applied to other spontaneous events for other psychological states, such as craving associated with an addiction. Manual or automated labeling may input into the AI model to optimize AI algorithm efficiency and/or accuracy.

Some AI models may not use, or need, labeled data.

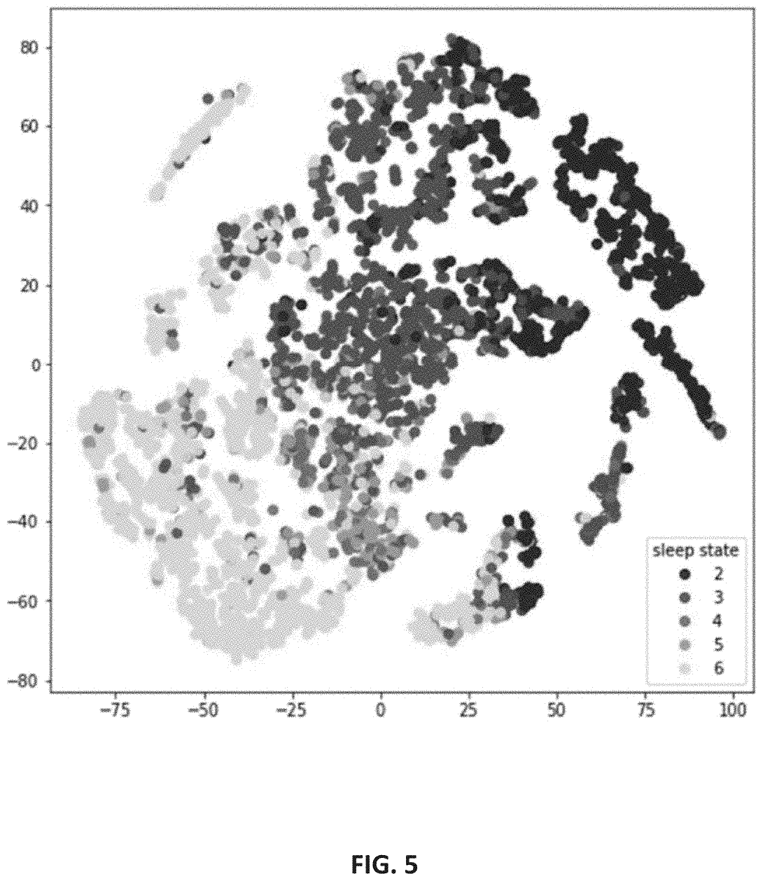

In general, an AI model, such as a DNN, may be trained to learn a representation of the data from which meaningful tasks may be subsequently performed. These representations, some of which may be associated with particular psychological brain states, most likely will not be human understandable due to their high dimensionality. Lower dimensional projections of the data, such as the tSNE plot in FIG. 5, may be employed to visualize the learned representations and clusters corresponding to psychological brain states. The learned representation of the data may be used to identify, classify, and/or predict one or more psychological brain states and/or predict the change to or from one or more different psychological brain states. In some embodiments, the AI model may be used to embed one or more features in the collected data to a representation space where the AI model may identify, classify, and/or predict.

Any suitable AI technique or combination of techniques may be used to establish or fine-tune the AI model. The AI model may be re-trained at irregular or regular intervals (e.g., daily, weekly, monthly) and may be changed and/or upgraded to integrate additional components, architectures, training methods, and other techniques. In some embodiments, the AI model may involve deep learning and a DNN. In some embodiments, the model may be trained through self-supervised learning. The self-supervised learning model may include one or more learnable parameters to facilitate learning and may include thousands, millions, or billions of learnable parameters. In some embodiments, the self-supervised learning model may be configured to learn a representation by predicting time between different segments of data. For example, three segments of data may be chosen randomly, but in such a fashion that two of the segments are close together in time and the third is further away. The model may then be trained to embed the two close in time segments to points in representation space that are close to one another, and far away from the third segment. Such triplets of time segments may be automatically generated from unlabeled data. In another example, the model may be trained to predict or impute values in the EEG data that have been intentionally removed. These values may be short time segments of data, data collected from certain electrodes, or a combination thereof.

In some embodiments, the AI model is trained using supervised machine learning. In particular, supervised machine learning may be used when the collected data is labeled. The subject, a physician, a sensor, or the like may be used to label data by temporally marking a psychological brain state relative to concurrently collected EEG data, and the AI model may be trained to classify the psychological brain state corresponding to a segment of labeled EEG data.

In other embodiments, the AI model is trained using unsupervised machine learning. For example, unsupervised machine learning may be used when the collected data is unlabeled. Relevant features in the collected data may be automatically learned and extracted using unsupervised learning, and clusters corresponding to psychological brain states may emerge naturally using these techniques. In general, the unlabeled collected set of data may be much larger than a labeled set of data to appropriately train the AI model. In still other embodiments, the AI model may use semi-supervised learning. In one example, only some of the collected data may be labeled, in which case the model may first be trained with unsupervised or self-supervised techniques and then trained, subsequently or concurrently, with the labeled data.

Transfer learning may also be used to train the AI model. In some embodiments, the AI model may first be trained on a large set of unlabeled data, such as EEG data of one or more subjects, or perhaps data from non-EEG domains. The AI model may subsequently be trained on unlabeled or labeled EEG data to fine-tune the AI model, for example, for use on one particular subject and a particular task.

Unsupervised, self-supervised, or supervised learning may benefit from using very large volumes of diverse data to train the AI model. For example, iEEG may be used to provide the diverse data. Subsequent transfer learning may benefit from using robust feature extractors trained on very large volumes of diverse data.

The AI model may be further trained using transfer learning based on a different data set, such as EEG data collected from different sensors. In one example, the AI model may first be trained based on iEEG data collected from one or more electrodes implanted in one or more subjects. The model may then be trained on, for example, 7-day scalp-based EEG or MEG data from a subject. This approach may be useful for identifying, classifying and/or predicting psychological brain states in subjects without implants using an AI model initially trained on data from subjects with implants.

Data other than iEEG may be used by the AI model to identify, classify, and/or predict a psychological brain state. In some embodiments, other data used to supplement the EEG data may include one or more of the following types of data corresponding to the subject: activity, motion, heart rate, visual stimuli, audio or video recordings, caregiver assessment, observer assessment, and skin conductance.

These additional data sources may similarly be used by the AI model to develop a robust feature extractor as part of a pretext task. In some embodiments, EEG data may be used by the AI model to predict the state or states of the other data.

In some embodiments, methods may include an iterative process in which data (e.g., EEG data) is continually collected and the AI model is refined by further training. Over time, the AI model may improve. In particular, the virtuous cycle of a trained model may continue to extract features related to events over time that identify and predict events more accurately as more data is provided to the model for training. Accuracy of the AI model may increase over time to more accurately identify or predict a psychological brain state based on the collected data.

In some cases, labeled data may be collected in an artificial experimental setting. Sequential psychological brain state triggers may be provided and time identified and may occur in one setting or over multiple settings. Once a base number of specific triggers and consequent psychological brain state responses are created, an AI model may be trained that uses measured time triggers and then tested on data not used for training to determine if the created AI model can predict psychological brain states in the experimental setting. Afterwards, the AI model may be tested outside of the laboratory or experimental setting to determine if psychological brain states can be predicted in a real-world non-experimental setting. Any suitable base number of specific triggers may be employed. For example, a base number of 5 or more triggers, 10 or more triggers, 20 or more triggers, 30 or more triggers, 40 or more triggers, or 50 or more triggers may be used. The base number of triggers may be used to start the data collection approach and then increased from there. An example of a trigger may be a visual stimulus that triggers a traumatic memory in a subject suffering from PTSD. For example, military personnel suffering from PTSD may be shown an image of a military setting that trigger an emotional aspect of PTSD to trigger an emotional state. In a single experimental sitting, a limited number of triggers may be applied. Accordingly, multiple experimental sittings may be needed to obtain the base number of triggers.

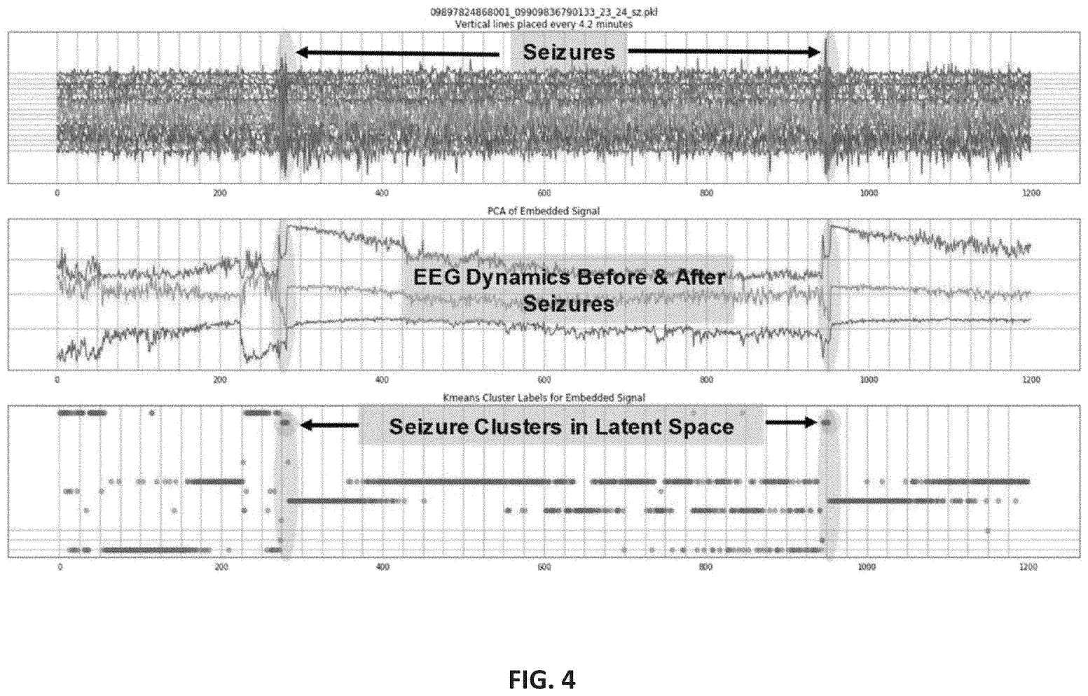

The AI model may be further trained based on the labeled data. In one example, raw data may be provided to a deep neural net with a self-supervised pretext task. FIG. 4 shows, for purposes of illustration, one example of a lower-dimensional view of a representation space and cluster arrangement provided by a self-supervised deep neural network trained on raw, unlabeled EEG data. The view is provided as a lower-dimensional because higher dimensional data, while readily manageable in an AI model, may not be human understandable. FIG. 4 illustrates EEG data over time and unsupervised clusters of similar data provided by a self-supervised deep neural network trained on raw, unlabeled EEG data, for seizure identification, classification, and prediction. This illustration is merely for purposes of example, as identification, classification, and/or prediction of epileptogenic brain activity is not identification, classification, and/or prediction of a psychological brain state. FIG. 5 shows, for purposes of illustration, a 2-D visualization of an unsupervised representation space, colored by sleep stages. More specifically, FIG. 5 illustrates output of a tSNE algorithm run on an embedding of EEG data taken from a latent space in a deep neural network. Like FIG. 4, FIG. 5 is shown merely for purposes of example and is an illustration of how different psychological brain states—in this case differing sleep stages—cluster in different regions of latent space.

FIGS. 4 and 5 attempt to illustrate examples of how AI models may separate different psychological brain states (in this case sleep stages and epileptogenic events). Most importantly, the deep neural network used in these examples was trained with self-supervision on a large quantity (i.e., months) of iEEG without using data labels. The neural network in this case was not trained for a particular classification or a prediction task, but rather to be a general EEG feature extractor network. The clusters of similar psychological brain states arose naturally by processing large quantities of data through the trained feature extractor network because the same psychological brain state measured at different times produce very similar iEEG features. These clustering examples demonstrate that these models may be fine-tuned with few labels to develop a classification model for sleep states and/or a predictive model for epileptogenic events. As such, developing an AI model capable of discriminating broad categories of brain states with few labeled examples is feasible. FIGS. 4 and 5 also serve to illustrate that AI may be used to predict events other than psychological brain states based on brain activity data and that the use of brain activity data to identify, classify, and/or predict a psychological brain state through an AI model is feasible.

One approach to self-supervised learning may utilize a large number (e.g., thousands, millions, billions or more) of automatically generated labels, which may be used to train a feature extractor with a pretext task. Such an approach was taken for the models in FIGS. 4 and 5. For example, one channel (e.g., from a first electrode) may be used to predict features in another channel (e.g., from a second electrode). Further, limited labeled data may be used to fine-tune the AI model.

In embodiments described herein, a method comprises collecting data, such as EEG data, utilizing an established AI model, which may be an AI model that is continuing to be refined, to output data regarding a current or future psychological brain state based on the collected data. The output data may be used for any suitable purpose. For example, the data may be output to warn a subject that an adverse event may occur in the near future based on a predicted psychological brain state. For example, if the subject is suffering from post-traumatic stress disorder (PTSD), the subject may receive a warning that a sense of anxiety may soon occur. The data may be output to inform a third party, such as a healthcare provider or caretaker, that an adverse event is occurring or has occurred.

The AI model may be developed in any suitable system. The AI model may be installed in any suitable system. In some embodiments, the AI model is developed and/or trained in a system remote from a subject. In some embodiments, the AI model is installed in a system that is associated with the subject, such as in an implantable device, an ambulatory device, a wearable device, or a mobile computing device.

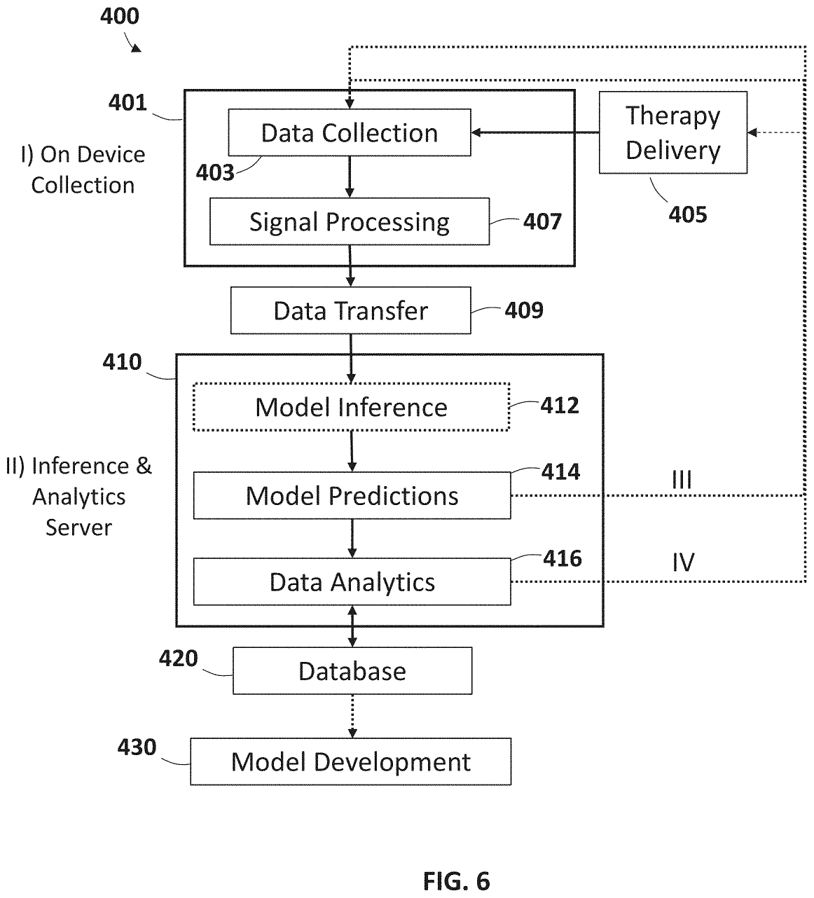

Referring now to FIG. 6, a method 400 for deploying an AI model for identifying, classifying, and/or predicting a psychological brain state is shown. The illustrated method shows therapy delivery in block 405, but it will be understood that the AI models may be used for any suitable purpose other than therapeutic purposes. The depicted deployment process includes an on-device collection component (401) and an inference and analytics server component (410). Brain activity data, such as iEEG data may be collected in real-time by collection component (401), which may include one or more implanted intracranial electrode and associated signal apparatus for collecting the data, as shown in block 403. Signal processing may be applied to the collected data as shown in block 407. The signal processing may include, for example, application of one or more filters. The processed data may then be transferred off device as illustrated in block 409 and transferred to the model inference and analytics server (410). Any suitable model inference and analytics server (410) may be used. For example, the model inference and analytics server (410) may be a mobile computing device, an on- or off-site computer, a cloud computing instance and/or a combination thereof. To the extent that it has sufficient processing power, the collection and therapy component (401) may include or be the model inference and analytics server (410).

The data may be then further pre-processed and fed through a trained AI model as shown in block 412. The model outputs classifications and/or predictions of psychological brain states or predicts psychological brain state changes as shown in block 414. The model's predictions may be transferred back to the collection component (401), as illustrated by line III. Alternatively, the model's predictions may be analytically combined with historical model predictions, as shown in block 416, and then transferred back as shown by line IV. The resultant output may be used to inform the delivery and/or the adjustment of therapy as shown in block 405. Data collection (401) may be performed before, during, and after therapy delivery (405) or therapy adjustments, and the model may be used to aid in determining whether the therapy delivery (405) or adjustments to therapy delivery (405) are effective. Data regarding the therapy delivery (405) may be collected as labeled data and may be used by the model in conjunction with brain activity data collected in block 403. Data regarding therapy delivery (405) may include timing of delivery, dosing, and the like.

In most embodiments, the total time from block 403 to back to block 403 (e.g., through blocks 407, 409, 412, 414, and 416) is near-real-time; i.e., all processes and pathways are preferably optimized to reduce latency.

Data from the device (401) and outputs from the inference and analytics server (410) may be ingested into a database as shown in block 420 for long term storage and review (e.g., by a physician). These data may be used in a virtuous cycle to re-train and upgrade the model as shown in block 430 at regular and/or irregular intervals. The model may be re-trained or upgraded in any suitable manner. These data may also be subsequently used by the inference and analytics server to analytically combine future model predictions with historical data, as shown in block 416. In some embodiments, the model may be re-trained and/or upgraded as described above regarding FIG. 3.

An established AI model may be installed on any suitable device that is in communication with apparatus transmitting EEG data (e.g., the signal apparatus 620 or external device 500 depicted in, and discussed above, regarding FIG. 2) or other suitable brain activity data, such as MEG data, so that the device on which the AI model is installed may apply the AI model to signals as they are received by the electrodes recording the EEG signals or sensors recording other suitable brain activity data. The device on which the AI model is installed may provide output regarding a current or predicted future brain state based on data regarding the signals recorded by the electrodes.

Once an AI model has been established using iEEG data, the AI model may be fine-tuned and/or applied to brain activity data obtained from, for example, scalp EEG or MEG devices. The AI model may be applied to other subjects and other EEG modalities. That is, subjects that do not have, or did not have, recording electrodes implanted in or on their brain. The transferred AI model may be installed on any suitable device that is in communication with apparatus transmitting the scalp-based EEG or MEG data.

In some embodiments, output data may be employed to administer therapeutic treatment to a subject suffering from a brain disease. In particular, the treatment may be administered in response to a current or predicted psychological brain state determined by the AI model. In some embodiments, treatment may be administered using a device, drug, or combination thereof. In one example, treatment may be administered by providing an alert through a device to the subject or to a physician. In another example, treatment may be administered by infusing a drug, removing fluid, or providing electrical deep brain stimulation to the subject.

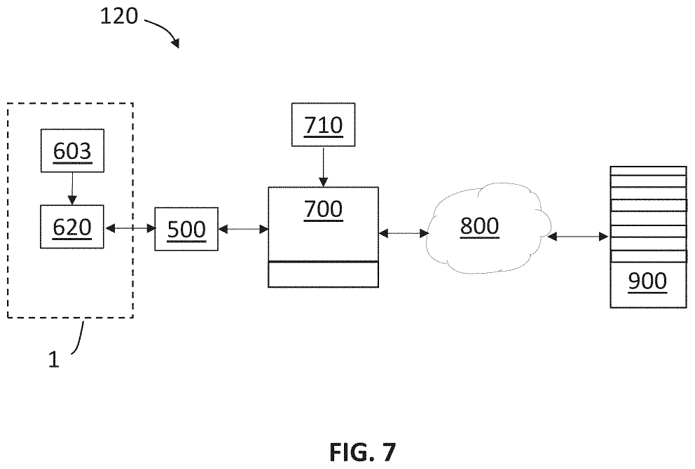

FIG. 7 shows one example of a system 120 that may be used with a method as described herein to collect EEG data and identify, categorize, and/or predict a psychological brain state based on the collected EEG data through the use of an AI model. While the distribution of components in FIG. 7 may be slightly different than the distribution shown in FIG. 6, the system 120 depicted in FIG. 7 may be configured to carry out a process generally as depicted in FIG. 6.

The system 120 in FIG. 7 includes a recording electrode 603 and signal apparatus 620, which may be implanted in a subject 1. If an AI model has already been developed based on iEEG data, the recording electrodes 603 and signal apparatus 620 may be external to the subject 1 (e.g., a scalp-based EEG system or a MEG system). The system 120 may include an external device 500 configured to communicate with the signal apparatus 620. The external device 500 may receive data transmitted from the signal apparatus 620 regarding data recorded by the recording electrodes 603. The external device 500 may be as described regarding FIG. 2 above. The external device 500 may transfer data received from the signal apparatus 620 to a secondary device 700. The secondary device 700 may be a laptop computer, desktop computer, tablet, smart phone, or the like. In some embodiments, the signal apparatus 620 may transmit data directly to the secondary device 700. The secondary device 700 may send data to server 900, for example, through the internet 800. In some embodiments, the signal apparatus 620 or the external device 500 may transmit data to the server 900, for example, through the internet 800. The AI model may be trained, make classifications and/or predictions regarding psychological brain states, or otherwise analyze brain activity data (e.g., as described with regard to blocks 412, 414, and 416 in FIG. 6) on the server 900. Alternatively or in addition, one or more aspects of AI model training, psychological brain state classification and/or prediction, and data analytics may be performed on the signal apparatus 620, the external device 500, and the secondary device 700.

The system 120 of FIG. 7 includes an input device 710, which may provide input to the system regarding labeling data. For example, the input device 710 may communicate with secondary device 700, which may send data labels to the server 900, e.g., through the internet 800. The input device 710 may send data labels to the server 900 (e.g., through the internet 800) or may be operably coupled to the external device 500, signal apparatus 620, or any other suitable device to provide data to the server 900 to develop, train, or fine-tune the AI model. Any suitable input device 900 may be used. The input device 900 may be a stand-alone device, such as an activatable button operably coupled to suitable data transmission components, may be a smart phone, tablet, or computer touchscreen, where the smart phone, tablet, or computer runs an application responsive to touch screen input and capable of transmitting data labels resulting from the touch screen input, or the like.

The server 900 may output data regarding an identity, classification, and/or prediction regarding a psychological brain state based on the data provided to the server 900. The output data may be received by any one of the secondary device 700, the external device 500, the signal apparatus 620, or any other suitable device. The output data may be provided to the subject, the subject's physician or caregiver, or the like. If the subject is undergoing treatment for a disease that is related to the psychological brain state, the physician, caregiver, or patient, may adjust therapy based on the data regarding the identity, classification, and/or prediction regarding the psychological brain state.

Specific treatments may be selected based on the disease being treated.

The treatment may be provided in a continual (or periodic), reactionary, or preventative manner. Treatment may be administered continually to prevent onset of a potential psychological brain state in a continual manner. For example, treatment may be delivered according to a schedule. Classification and forecasting analytics may not be needed for continual treatment but monitoring for toxicity may be increased.

Treatment may alternatively be administered upon onset of a psychological brain state in a reactionary manner. Event classification analytics may be used to provide reactionary treatment. Forecasting may not be needed. Certain undesirable psychological brain states may be shorted but may not be prevented when using reactionary treatment.

Further, treatment may be administered to the subject only, or selectively, before onset of a predicted psychological brain state of the subject in a preventative manner. Forecasting may be used to provide predictive treatment. The need for toxicity monitoring may be decreased. Certain undesirable psychological brain states may be prevented or avoided by using predictive treatment.

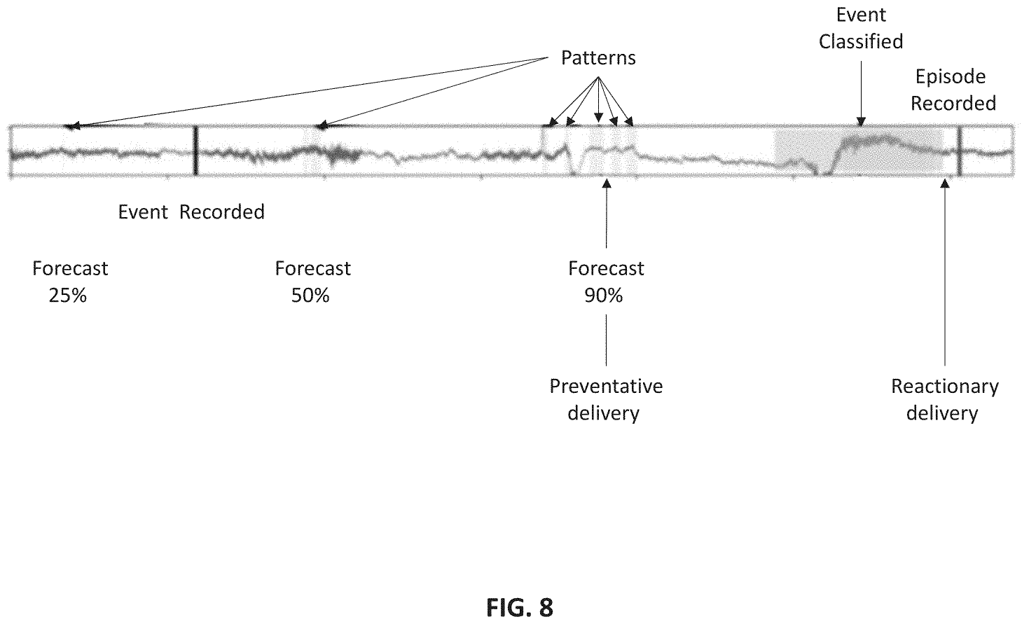

One example of the relationship between brain activity data patterns, psychological brain states, and different types of treatment is schematically shown in FIG. 8. Some brain activity data patterns may be associated with a higher probability of forecasting a psychological brain state than other brain activity data patterns (e.g., 90% vs. 50% vs. 25% forecast) according to the AI model. In addition, as more data is provided to an AI model the ability of the model to forecast a psychological brain state improves. The AI model may progressively improve prediction capability once a threshold prediction capacity has been reached due to re-training with additional labeled or unlabeled data. Thus, an “established” or “developed” AI model may continue to be trained and refined as more data is provided to the model. Brain activity data patterns understandable by humans may be identified in some embodiments, but not in others. In cases where human understandable brain activity data patterns are not identified, the AI model may make predictions directly from data representations without a human interpretable reason.

In some embodiments, human understandable brain activity data patterns corresponding to a current, past, or future brain state may be identified by the AI model with high sensitivity and specificity. In such cases, the brain activity data patterns may be referred to as “biomarkers” of the psychological brain state. Existing AI visualization techniques from other domains, such as language processing or computer vision, may be employed to identify such brain activity patterns; e.g., class activation mapping, attention visualization, and similar techniques.

The AI model may provide classifications or predictions to a subject, caregiver, or physician so that therapy may be administered or adjusted based on the classifications or predictions. Therapy may be delivered based on output from the AI model in a preventative (before the event is classified and based on a prediction) or reactionary (after the event is classified) manner.

AI models may be used to determine or predict any suitable psychological brain state. Examples of suitable psychological brain states include motivation, memory, attention, concentration, mood, cognition, happiness, arousal, fear, depression, elation, joy, confident, satisfied, focused, challenged, interested, in love, flow (a mental state in which a person becomes fully immersed in an activity), obsession, dream, fantasy, suicidal, hallucination, stress, anxiety, and the like, and degrees thereof.

Preferably, the data for developing the AI model is obtained from intracranial electrodes that are implanted during a medically warranted procedure in which the subject's brain is accessed, such a brain surgery, ventriculostomy, implantation of a therapeutic catheter or lead, craniotomy, or the like. The AI model may be developed for any suitable brain state. The brain state may be a brain state associated with a disease being treated or may be a non-disease brain state.

For some brain diseases, a subject may present with an extreme brain state. For example, a subject suffering from PTSD may experience extreme anxiety when recalling a traumatic memory. While not intending to be bound by theory, it is believed that extremes of psychological brain states may be associated with more pronounced brain activity patterns that may be more readily detectable by AI models. iEEG data associated with such extreme psychological brain states may facilitate initial AI model development and training. Once an AI model is established based on iEEG data associated with an extreme psychological brain state, the AI model may be transferred to iEEG data associated with less extreme psychological brain states, which may have less pronounced brain activity signals or patterns. For example, an AI model may be developed and trained on iEEG data from one or more subjects suffering from PTSD and who experience extreme anxiety during traumatic memory recall. Once an AI model for extreme anxiety is established, the AI model may be transferred and fine-tuned on iEEG data associated with more generalized anxiety from the same subject or subjects or another subject or subjects.

Alternatively, a general iEEG feature extractor model may be trained using unsupervised or self-supervised methods and large quantities of unlabeled iEEG from one or more subjects, such as the models developed to produce FIGS. 4 and 5. These models may be fine-tuned using iEEG labeled for specific brain states from the same subject or subjects or another subject or subjects.

General iEEG feature extractor models or extreme or less extreme psychological brain state AI models, regardless of whether based on a disease state, may be transferred to data received by more noisy signals obtained from non-invasive sources, such as scalp-based EEG, MEG, or the like; and fine-tuned for the non-invasive source domain.

As another example, an AI model trained to classify and/or predict elation or mania in bipolar disorder may be fine-tuned to develop an AI model for happiness. That is, the AI model for elation or mania may be transferred and fine-tuned on brain activity data (iEEG, scalp-based EEG, MEG, or the like) associated with happiness.

As another example, an AI model trained to classify and/or predict depression, despair or hopelessness may be fine-tuned to develop an AI model for mild depression. In some embodiments, one or more AI models may be trained to classify and/or predict multiple mood states along a continuum, such as despair, depression, content, happiness, and elation. Another continuum as an example is an attentional continuum, with the severe situation being severe attention deficit disorder and a mild example being clinically insignificant inattentiveness. Another example is gambling obsessional disorder or internet obsessional disorder or alcoholism or substance abuse disorder with a more mild version of that inconvenient occasional temptations or splurging thoughts. Another example is Post Traumatic Memory Disorder with frequent recollection of traumatic disabling memory with on the more mild side infrequent distressing memory recall that recalls an unpleasant memory from childhood.

Another example of degrees of psychological brain states that may facilitate development of AI models initially with an extreme and then with a milder degree includes extreme focus or flow, deep thinking, and engaged in an activity. In some embodiments, an AI model may be trained to classify and/or predict this kind of thought associated with maximization of productivity and contentment in work and study. An AI model may used also for meditation training to enable the brain to relax and focus and benefit from feedback on such a trained thinking approach.

AI models that can determine or predict a psychological brain state may be used for any suitable purpose. For example, the AI models may be used in treating subjects suffering from disease, particularly psychological and psychiatric diseases. The AI model may be used to determine if therapy is effective or to determine if one therapy is more effective than another. For example, the AI model may be employed to determine whether one or more of the frequency, duration, or severity (degree) of a psychological brain state associated with the disease decreases, which would indicate that the therapy is effective. Such a decrease may be, for example, part of the natural history or may be changed by psychological treatment, medical treatment, or a particular individual behavior.

PTSD is one example of a disease that may benefit from use of an AI model to identify or predict a psychological brain state. A number of disease-related psychological brain states are associated with PTSD and include extreme anxiety, suicidality, addiction, obsession or lack of impulse control, agitation, and cognitive dysfunction. Monitoring such brain states and determining whether psychological therapy or a medical therapy is effective in reducing the frequency, severity, or duration of such brain states may be beneficial. The ability to predict whether a subject suffering from PTSD will have suicidal thoughts may permit intervention before the thoughts occur.

The standard of care (SOC) for severe PTSD includes psychotherapy and minimal medications. A new medication and direct intervention may be added to SOC for improvement in certain symptoms captured in the Clinician-Administered PTSD Scale (CAPS) symptom scale, primarily B symptoms. An established AI model may be fed brain activity data (e.g., ciEEG, iEEG, EEG, MEG, or the like) from the subject suffering from PTSD to demonstrate improvement in such specific symptoms.

An established AI model tied to CAPS symptoms may be used to provide an early indication of therapy treatment effect, provide information on amount, need and timing of dose titration, and facilitate “Go”/“No Go” therapy decisions for personalization of oral medication and behavioral therapy including the SOC. In general, refractory PTSD subjects who suffer from repetitive distress caused by traumatic memory recall, flashbacks, and sleep disorders may be helped using the AI model. This approach and pattern of addressing spontaneous distressing events may also be applied to panic disorder which has a similar time course.

Traumatic brain injury (TBI), which may be associated with increased intracranial pressure (ICP) during the initial phase of injury, is another example of a disease that may benefit from use of an AI model to identify, quantify, or predict a psychological brain state through the treatment continuum. Many subjects suffering from TBI experience during the ensuing, months weeks, and years suffer from anxiety, suicidal ideation, agitated behavior, lack of attention, cognitive impairment and may suffer from PTSD as well. Monitoring such brain states and determining whether therapy is effective in reducing the frequency, severity, or duration of such brain states may be beneficial.

If the frequency, severity, or duration of an adverse psychological brain state is not reduced by a first therapy for a particular disease, a second therapy may be administered to the subject. The effectiveness of the second therapy may be monitored by determining whether the frequency, severity, or duration of an adverse psychological brain state is reduced or increased.

AI models that can determine or predict a psychological brain state may be used to enhance human performance. For example, the AI models may be used to enhance decision making, for education training, for career training, for learning optimization, for fear management, for impulse control, or the like. For example, AI models may identify, quantify or predict psychological states such as attention and flow based on brain activity data from a subject to enhance efficiency, focus, and productivity of the subject. AI models may identify, quantify or predict psychological states such as cognitive bias and dissonance based on brain activity data from a subject to enhance accuracy of the subject. AI models may identify, quantify or predict psychological states such as anxiety based on brain activity data from a subject to enhance confidence, collaboration, or leadership of the subject.

AI models may be trained on brain activity data from experts or exemplary individuals to identify or quantify psychological brain states of such individuals to help aid non-experts to achieve such brain states. Examples of such experts may include military officers, pilots, persons with access to nuclear codes, and surgeons.

AI models that can determine or predict a psychological brain state may be used to tailor consumer advertising. AI models may determine whether a subject is experiencing a positive (e.g., happy) or negative (e.g., sad) psychological brain state associated with an advertisement based on brain state activity of the subject while viewing or listening to the advertisement. The advertisement may be modified until the desired psychological brain state is elicited in a threshold percentage of subjects while viewing or listening to the advertisement.

AI models that can determine or predict a psychological brain state may be used to enhance gaming experiences. For example, AI models may determine whether a subject is experiencing anxiety or excitement based on brain state activity of the subject while playing a computer game. The data regarding the subject's psychological brain state or states may be feedback to the gaming software, which may modify gaming parameters to modify (e.g., enhance, diminish, or change) the subject's psychological brain state or a degree thereof.

Other uses of the AI models include military, law enforcement, and intelligence applications including readiness training, personnel placement and monitoring, leadership selection, and the like. The AI models may be used as a modern lie detector, for ethics training validation, to determine level of empathy, and the like.

Provided below are some brief examples of how AI models may be established and trained and applied. Examples include PTSD, bipolar disorder, suicidal feelings or thoughts, and flow.

Brain electrical data from a subject suffering from PTSD may be used to train an AI model. The subject may recall a traumatic memory and may experience sadness, anxiety, or dysphoria. The subject may press a button that is wirelessly connected to the subject's smartphone (e.g., using BLUETOOTH™) or may press a button displayed on a screen of the subject's smartphone. The button press may indicate a psychological brain state, or event, that is timestamped relative to the collected brain activity data. Data and time regarding the button press (constituting labeled data) may be used to train the AI model. After many events with time before and after monitored, for example 40 of these events, sufficient accuracy may be obtained to utilize the AI model outside the experimental setting, such as in a real-world therapeutic setting. The identification of the pattern may also facilitate analysis of pre- and post-event neuronal circuitry. By simple or complex manipulation of the brain electrical data, data may be augmented for training to enhance performance of the AI model. Data from patients who experience abnormally frequent episodes of a chosen psychological brain state (for example traumatic memory recall) may be chosen for efficient AI model training; however, care must be taken to ensure data from these patients is representative of the broader population.

Another example of conditions, disorders, or diseases, that may be approached using the AI model is bipolar disorder, which has a different time course and pattern than PTSD. The symptom of mania is of a longer duration than a memory flashback and the onset of the mania more typically increases relative slowly over time. Similar to a memory flashback in PTSD, subjects who are having their feelings of elevated mood, increasing confidence, rush of thoughts, increasing wanting to take risks (aspects of mania), may push a button to mark or label data. EEG and feature extraction will proceed using the AI model in a similar way and will preferably reach a threshold number of events. Once an AI model is trained to classify and/or predict one psychological brain state, it may be fine-tuned to classify and/or predict different psychological brain states (i.e., transfer learning). This may be done so that the AI model learns a new task at the expense of no longer being able to perform the initial task, or so that the AI model learns the new task while remaining proficient at the prior task. For example, once trained, the AI model may be used to classify and/or predict bipolar disorder, and subsequently classify and/or predict to treat depression and generalized anxiety disorders, or states, in a similar manner.

Yet another example of conditions, disorders, or diseases, that may be approached using the AI model is suicidal feelings and thoughts. While the time course and nature of thoughts are different and outcomes are different, a similar approach can be followed as described herein with respect to PTSD and bipolar disorder. Subjects may have spontaneous thoughts of suicide and spontaneous thoughts of compulsion to act on suicide or to not have insight of the temporariness of these obsessive thoughts and act upon them. When subjects have thoughts spontaneously, the thoughts may be characterized with stamping and signaling (e.g., marking and labeling). Over time, the EEG recognition will be discerned though AI model. Patients who may be selected to train the AI model may be those with more frequent suicidal thoughts or obsessions than is common in the general population, as brain electrical data from such patients may more efficiently train the AI model due to the frequency of the occurrences. Timely medical or social or other intervention may be enabled using this technique, which may help abort a suicide attempt or a completed suicide. Such an approach may also be used to treat addiction disorders, including for alcohol and cocaine as well as those involving obsessive thoughts like internet and pornography and compulsive checking or thoughts amongst others, in a similar manner.

Normal psychological events, such as flow (e.g., as described by Mihaily Csikszentmihalyi in for example, Flow: The Psychology of Optimal Experience (Jul. 1, 2008), Harper Collins Publishers) may be identified. Flow is a brain state associated with optimal attention, satisfaction, and happiness that occurs when engaged in challenging and stimulating activities overlapping with attention and learning. The subject may have the ability to recognize the state and mark the events and after enough events are identified in the EEG, and feature extraction in the AI model may be used to identify, quantify and afterwards predict brain states or events. Such an approach may not be restricted to flow, but may include other, learning and decision-making brain states and processes.

One or more of the components, such as controllers, sensors or interfaces, described herein may include a processor, such as a central processing unit (CPU), a graphics processing unit (GPU), computer, logic array, or other device capable of directing data coming into or out of a device, system, or component thereof. The controller may include one or more computing devices having memory, processing, and communication hardware. The controller may include circuitry used to couple various components of the controller together or with other components operably coupled to the controller. The functions of the controller may be performed by hardware and/or as computer instructions on a non-transient computer readable storage medium.

The processor of the controller may include any one or more of a microprocessor, a microcontroller, a digital signal processor (DSP), an application specific integrated circuit (ASIC), a field-programmable gate array (FPGA), and/or equivalent discrete or integrated logic circuitry. In some examples, the processor may include multiple components, such as any combination of one or more microprocessors, one or more controllers, one or more DSPs, one or more ASICs, and/or one or more FPGAs, as well as other discrete or integrated logic circuitry. The functions attributed to the controller or processor herein may be embodied as software, firmware, hardware, or any combination thereof. While described herein as a processor-based system, an alternative controller could utilize other components such as relays and timers to achieve the desired results, either alone or in combination with a microprocessor-based system.

In one or more embodiments, the functionality of systems and devices may be implemented using one or more computer programs using a computing apparatus, which may include one or more processors and/or memory. Program code and/or logic described herein may be applied to input data/information to perform functionality described herein and generate desired output data/information. The output data/information may be applied as an input to one or more other devices and/or methods as described herein or as would be applied in a known fashion. In view of the above, it will be readily apparent that the controller functionality as described herein may be implemented in any manner known to one skilled in the art.

While the present disclosure is not so limited, an appreciation of various aspects of the disclosure will be gained through a discussion of the specific examples and illustrative embodiments provided below. Various modifications of the examples and illustrative embodiments, as well as additional embodiments of the disclosure, will become apparent herein.

Example 1: A method comprising transmitting, for a long duration of time, continuous EEG data derived from at least one intracranially implanted electrode to an external device.

Example 2: The method according to Example 1, further comprising intracranially implanting the at least one electrode in a subject during a medically warranted procedure in which the subject's brain is accessed.

Example 3: A method comprising receiving, by an external device and for a long duration of time, EEG data derived from at least one intracranially implanted electrode. The received EEG data may be broad filtered, narrow filtered, or band filtered or unfiltered.

Example 4: A method comprising (i) receiving, for a long duration of time, continuous EEG data derived from at least one intracranially implanted electrode for a long duration of time; and (ii) establishing an artificial intelligence (AI) model to identify, classify, and/or predict a psychological brain state of the subject based on the EEG data. The received EEG data may be broad filtered, narrow filtered, or band filtered or unfiltered.

Example 5: The method of Example 4, wherein the continuous EEG data is received by an external device.

Example 6: The method of any one of Examples 1 to 5, wherein the continuous EEG data is transmitted or received continuously for a duration of time that extends over at least 5 psychological brain state episodes.

Example 7: The method of any one of Examples 1 to 5, wherein the continuous EEG data is transmitted or received continuously for a duration of time that extends over at least 10 psychological brain state episodes.

Example 8: The method of any one of Examples 1 to 5, wherein the continuous EEG data is transmitted or received continuously for a duration of time that extends over at least 40 psychological brain state episodes.

Example 9: The method of any one of Examples 4 to 8, further comprising labeling the EEG data according to one or more of time, duration, and occurrence of a psychological brain state, and wherein establishing the AI model comprises using the labeled EEG data labeled to train a developing AI model via supervised machine learning.

Example 10: The method of any one of Examples 4 to 9, further comprising labeling the EEG data according to one or more of time, duration, and occurrence of a psychological brain state, and wherein establishing the AI model comprises using a combination of the labeled and unlabeled EEG data to train a developing AI model via semi-supervised machine learning.

Example 11: The method of any one of Examples 4 to 10, wherein establishing the AI model comprises using unlabeled EEG data to train a developing AI model using unsupervised learning.

Example 12: The method of Example 11, wherein establishing the AI model comprises fine-tuning the AI model that has been previously trained using different data and/or a different task via transfer learning.

Example 13. The method of Example 12, wherein the AI model had been previously trained with one or more of supervised, unsupervised, self-supervised and semi-supervised learning, and wherein the fine tuning comprises employing one or more of supervised, unsupervised, self-supervised and semi-supervised learning methodologies.

Example 14: The method of any one of Examples 4 to 13, further comprising (i) receiving electroencephalogram (EEG) or magnetoencephalogram (MEG) data from a device positioned on the scalp or under the scalp of the subject; and (ii) training the established AI model with the EEG or MEG data from the device positioned on the scalp or under the scalp of the subject to identify, classify, and/or predict the psychological brain state in the EEG or MEG data from the device positioned on the scalp or under the scalp of the subject.

Example 15: The method of any one of Examples 4 to 13, further comprising: (i) receiving electroencephalogram (EEG) or magnetoencephalogram (MEG) data from a device positioned on a scalp of a second subject different from the subject in which the electrode is implanted in or on the brain; and (ii) training the established AI model with the EEG or MEG data from the second subject to identify, classify, and/or predict the psychological brain state in the EEG or MEG data of the second subject.

Example 16: The method of any one of Examples 4 to 15, further comprising applying the established AI model to predict a future psychological brain state of the subject and/or to identify human understandable biomarkers in EEG data.

Example 17: The method of Example 16, comprising notifying the subject of the predicted future psychological brain state.

Example 18: The method of any one of Examples 4 to 17, comprising applying the AI model to adjust administration of a therapy to the brain of the subject or the second subject based on the received data.

Example 19: The method of Examples 4 and 18, wherein the AI model is trained to predict which of several EEG segments occur close together in time.

Example 20: The method of Examples 4 and 18, wherein the AI model is configured to generate and/or impute data that has been intentionally deleted from segments in time, data collected from other electrodes, or both.

Example 21: The method of Example 19 or 20, wherein the AI model is trained to predict the duration of time between two or more different segments of the EEG data.

Example 22: A method comprising: (i) receiving, for a long duration of time, continuous electroencephalogram (EEG) data from at least one electrode intracranially implanted in a subject; and (ii) determining a psychological brain state or predicting a future psychological brain state of the subject based on the EEG data using an artificial intelligence (AI) model.

Example 23: The method of Example 22, wherein the psychological brain state or predicted psychological brain state is associated with a disease of the brain of the subject.

Example 24: The method of Example 22 or 23, wherein the continuous EEG data is received by an external device.

Example 25: The method of any one of Examples 22 to 24, wherein the continuous EEG data is transmitted or received continuously for a duration of time that extends over at least 5 psychological brain state episodes.

Example 26: The method of any one of Examples 22 to 24, wherein the continuous EEG data is transmitted or received continuously for a duration of time that extends over at least 10 psychological brain state episodes.

Example 27: The method of any one of Examples 22 to 24, wherein the continuous EEG data is transmitted or received continuously for a duration of time that extends over at least 40 psychological brain state episodes.

Example 28: The method of any one of Examples 22 or 27, wherein the subject has or had a catheter implanted in the brain to treat a disease of the brain.

Example 29: The method of Example 28, wherein the catheter comprises an external ventricular drainage catheter or a ventriculoperitoneal shunt.

Example 30: The method of Example 28 or 29, wherein the catheter comprises a therapeutic agent delivery catheter.

Example 31: The method of Example 30, wherein the therapeutic agent delivery catheter is configured to deliver a therapeutic agent to a CSF-containing space of the subject.

Example 32: The method of Example 31, wherein the therapeutic agent delivery catheter is configured to deliver a therapeutic agent to a cerebral ventricle of the subject.

Example 33: The method of any one of Examples 22 to 32, wherein the at least one electrode is implanted in white matter or grey matter of the brain.

Example 34: The method of any one of Examples 22 to 33, wherein the at least one electrode is implanted in white matter of the brain.

Example 35: The method of any one of Examples 22 to 34, comprising administering treatment to the subject in response to the current or predicted brain state in the brain state data.

Example 36: The method of Example 35, wherein treatment is administered before onset of a predicted brain state of the subject.

Example 37: The method of Example 35 or 36, wherein administering treatment comprises providing an alert to the subject, a healthcare provider, or a caretaker.

Example 38: The method of any one of Examples 35 to 37, wherein administering treatment comprises infusing a drug, removing fluid, or electrical deep brain stimulation.

Example 39: The method of any one of Examples 35 to 37, wherein the treatment comprises a treatment for one or more of the following wherein the treatment comprises a treatment for one or more of the following: epilepsy, bipolar disorder, depressive disorder spectrum, anxiety disorder spectrum including PTSD, cognitive disorder spectrum, memory disorder spectrum, processing speed disorder spectrum subarachnoid hemorrhage, intracerebral hemorrhage, subdural hemorrhage, extradural hemorrhage, obstructive hydrocephalus, nervous system cancer (such as secondary malignant neoplasm of the brain, spinal cord, or other parts of the nervous system; malignant neoplasm of the cerebellum nos; malignant lymphoma of an unspecified site, extranodal site, or solid organ site; and the like), cerebral artery occlusion, unspecified cerebral infarction, closed fracture of the base of the skull (which may be associated with one or more of subarachnoid hemorrhage, subdural hemorrhage, extradural hemorrhage, and loss of consciousness), infection and inflammatory reaction (such as due to nervous system device, implant or graft), mechanical complication of a nervous system device, implant or graft), traumatic brain injury, congenital hydrocephalus, and aneurysms.

Example 40: The method of any one of Examples 22 to 39, wherein determining the psychological brain state or predicted future psychological brain state comprises receiving EEG data from two or more electrodes.

Example 41: The method of any one of Examples 22 to 40, comprising receiving other data than the EEG data and wherein the other data is used in determining the psychological brain state or predicting the future psychological brain state.

Example 42: The method of Example 41, wherein the other data comprises one or more of the following types of data corresponding to the subject: activity, motion, heart rate, visual stimuli, audio or video recordings, caregiver assessment, observer assessment, and skin conductance.

Example 43: The method of any one of Examples 22 to 40, comprising receiving data labels to label the psychological brain state relative to the EEG data and training the AI model based on the data labels and the EEG data.

Example 44: The method of any one of Examples 22 to 43, comprising training the AI model based on unlabeled EEG data via unsupervised learning.

Example 45: The method of any one of Examples 22 to 44, comprising training the AI model using EEG data labeled according to one or more of time, duration, and occurrence of a psychological brain states via supervised machine learning.

Example 46: The method of any one of Examples 22 to 45 comprising training the AI model using a combination of unlabeled EEG data and EEG data labeled according to one or more of time, duration, and occurrence of a psychological brain states via semi-supervised machine learning

Example 47: The method of any one of Examples 22 to 46, comprising fine-tuning the AI model that has been previously trained using different data and/or a different task via transfer learning

Example 48: The method of Example 47, wherein the AI model had been previously trained with one or more of supervised, unsupervised, self-supervised and semi-supervised learning, and wherein the fine tuning comprises employing one or more of supervised, unsupervised, self-supervised and semi-supervised learning methodologies.

Example 49: The method of any one of Examples 22 to 48, wherein the AI model is trained to predict which of several EEG segments occur close together in time.

Example 50: The method any one of Examples 22 to 49, wherein the AI model is configured to generate and/or impute data that has been intentionally deleted from segments in time, data collected from other electrodes, or both.

Example 51: The method of Example 49 or 50, wherein the AI model is trained to predict the duration of time between two or more different segments of the EEG data.

Example 52: The method of any one of Examples 4 to 51, wherein the AI model is able to identify human understandable biomarkers in the EEG data corresponding to a psychological brain state.

Example 53: The method of any one of Examples 1 to 52, further comprising implanting the at least one electrode in the subject while the subject is undergoing a surgical procedure to treat a disease of the brain.

Example 54: The method of Example 53, wherein the surgical procedure comprises inserting a catheter into the brain.

Example 55: The method of claim 54, wherein the catheter is a therapeutic fluid delivery catheter, a ventricular drainage catheter, or a catheter of a ventriculoperitoneal shunt.

Example 56: The method of Example 54 or 55, wherein the catheter comprises the at least one electrode.

Example 57: A device or system comprising one or more electrodes configured to be intracranially implanted in a subject to record electrical brain activity; and a controller operably coupled to the one or more electrodes configured to carry out the method according to any one of the Examples 4 to 52 or Examples 53 to 56 to the extent they depend from Examples 4 to 52.

Example 58: A device or system comprising one or more electrodes configured to be positioned on the surface of the head of a subject to record electrical activity of the white or grey matter of the brain of a subject; and a controller operably coupled to the one or more electrodes configured to carry out the method according any one of Examples 4 to 52 or Examples 53 to 56 to the extent they depend from Examples 4 to 52.

Example 59: The device or system of Example 57 or 58, wherein the controller is wirelessly coupled to the one or more electrodes.

Example 60: The device or system of any one of Examples 57 to 59, wherein the controller comprises at a remote computing system configured to train the AI model.

Thus, various embodiments of DEVELOPMENT AND IMPLEMENTATION OF PSYCHOLOGICAL STATE MODEL are disclosed. Although reference is made herein to the accompanying set of drawings that form part of this disclosure, one of at least ordinary skill in the art will appreciate that various adaptations and modifications of the embodiments described herein are within, or do not depart from, the scope of this disclosure. For example, aspects of the embodiments described herein may be combined in a variety of ways with each other. Therefore, it is to be understood that, within the scope of the appended claims, the claimed invention may be practiced other than as explicitly described herein.

All references and publications cited herein are expressly incorporated herein by reference in their entirety for all purposes, except to the extent any aspect directly contradicts this disclosure.

What is claimed is:

1. A method comprising:
transmitting, for a long duration of time, continuous EEG data derived from at least one intracranially implanted electrode to an external device.

2. The method according to claim 1, further comprising intracranially implanting the at least one electrode in a subject during a medically warranted procedure in which the subject's brain is accessed.

3. A method comprising:
receiving, by an external device and for a long duration of time, EEG data derived from at least one intracranially implanted electrode.

4. A method comprising:
receiving, for a long duration of time, continuous EEG data derived from at least one intracranially implanted electrode; and
establishing an artificial intelligence (AI) model to identify, classify, and/or predict a psychological brain state of the subject based on the EEG data.

5. The method of claim 4, wherein the continuous EEG data is received by an external device.

6. The method of claim 1, wherein the continuous EEG data is transmitted or received continuously for a duration of time that extends over at least 5 psychological brain state episodes.

7. The method of claim 1, wherein the continuous EEG data is transmitted or received continuously for a duration of time that extends over at least 40 psychological brain state episodes.

8. The method of claim 4, further comprising labeling the EEG data according to one or more of time, duration, and occurrence of a psychological brain state, and wherein establishing the AI model comprises using the labeled EEG data labeled to train a developing AI model via supervised machine learning.

9. The method of claim 4, further comprising labeling the EEG data according to one or more of time, duration, and occurrence of a psychological brain state, and wherein establishing the AI model comprises using a combination of the labeled and unlabeled EEG data to train a developing AI model via semi-supervised machine learning.

10. The method of claim 4, wherein establishing the AI model comprises using unlabeled EEG data to train a developing AI model using unsupervised learning.

11. The method of claim 10, wherein establishing the AI model comprises fine-tuning the AI model that has been previously trained using different data and/or a different task via transfer learning.

12. The method of claim 11, wherein the AI model had been previously trained with one or more of supervised, unsupervised, self-supervised and semi-supervised learning, and wherein the fine tuning comprises employing one or more of supervised, unsupervised, self-supervised and semi-supervised learning methodologies.

13. The method of claim 4, further comprising:
receiving electroencephalogram (EEG) or magnetoencephalogram (MEG) data from a device positioned on the scalp or under the scalp of the subject; and
training the established AI model with the EEG or MEG data from the device positioned on the scalp or under the scalp of the subject to identify, classify, and/or predict the psychological brain state in the EEG or MEG data from the device positioned on the scalp or under the scalp of the subject.

14. The method of claim 4, further comprising:
receiving electroencephalogram (EEG) or magnetoencephalogram (MEG) data from a device positioned on a scalp of a second subject different from the subject in which the electrode is implanted in or on the brain; and
training the established AI model with the EEG or MEG data from the second subject to identify, classify, and/or predict the psychological brain state in the EEG or MEG data of the second subject.

15. The method of claim 4, further comprising applying the established AI model to predict a future psychological brain state of the subject.

16. A method comprising:
receiving, for a long duration of time, continuous electroencephalogram (EEG) data from at least one electrode intracranially implanted in a subject; and
determining a psychological brain state or predicting a future psychological brain state of the subject based on the EEG data using an artificial intelligence (AI) model.

17. The method of claim 16, wherein the psychological brain state or predicted psychological brain state is associated with a disease of the brain of the subject.

18. The method of claim 16, wherein the subject has or had a catheter implanted in the brain to treat a disease of the brain.

19. The method of claim 18, wherein the catheter comprises the one or more electrode.

20. The method of claim 16, comprising administering treatment to the subject in response to the current or predicted brain state in the brain state data.
